(19) United States
(12) Patent Application Publication
(10) Pub. No.: US 2026/0061030 A1
(43) Pub. Date: Mar. 5, 2026

(54) COMPOSITIONS AND SYNERGISTIC METHODS FOR TREATING INFECTIONS

(71) Applicants: BioAegis Therapeutics Inc., Morristown, NJ (US); President and Fellows of Harvard College, Cambridge, MA (US)

(72) Inventors: Mark J. DINUBILE, Morristown, NJ (US); Susan L. LEVINSON, Morristown, NJ (US); Thomas P. STOSSEL, Morristown, NJ (US); Lester KOBZIK, San Carlos, CA (US)

(73) Assignees: BioAegis Therapeutics Inc., Morristown, NJ (US); President and Fellows of Harvard College, Cambridge, MA (US)

(21) Appl. No.: 19/384,043

(22) Filed: Nov. 10, 2025

Related U.S. Application Data
(63) Continuation of application No. 17/620,871.
(60) Provisional application No. 62/864,599, filed on Jun. 21, 2019.

(51) Int. Cl. A61K 38/17; A61K 45/00; A61P 31/04; A61P 31/12

(52) U.S. Cl. CPC A61K 38/1709; A61K 45/00; A61P 31/04; A61P 31/12

(57) ABSTRACT

The present invention relates to compositions and methods for treating microbial infections in subjects, in particular methods of administering a gelsolin agent and an antimicrobial agent to produce a synergistic therapeutic effect against a microbial infection in a subject. The present invention also relates to methods for treating viral infections in subjects, including methods that include delayed-dosing methods and/or synergistic methods.

### RELATED APPLICATIONS

This application claims the benefit under 35 U.S.C. § 119(e) of U.S. Provisional application Ser. No. 62/864,599 filed Jun. 21, 2019, the disclosure of which is incorporated by reference herein in its entirety.

### GOVERNMENT INTEREST

This invention was made with government support under grant NIH AI125152 and NIH/NIAID contracts HHSN272201000033I-HHSN27200003 and HHSN272201000033I-HHSN27200006. The United States government has certain rights in the invention.

### FIELD OF THE INVENTION

The invention, in some aspects, relates to compositions and methods for enhancing host immune defenses in the treatment of microbial infections.

### BACKGROUND OF THE INVENTION

Antimicrobial resistance is a worldwide public health concern. Antimicrobial resistance is known to reduce therapeutic efficacy of a variety of antimicrobial agents such as antibiotic agents, antiviral agents, antifungal agents, and antiparasitic agents. Examples of the evolving presence of resistant pneumococcal bacterial strains include: a case report of fatal resistant pneumococcal pneumonia (Waterer G W et al., Chest 2000; 118:1839-1840) and a finding that 22% (139/643) patients hospitalized for S. pneumonia had macrolide-resistant organisms (Cilloniz et al., Am J Respir Crit Care Med 2015; 191:1265-1272). Recent publications evidence (1) resistance of S. pneumoniae isolates from invasive infections to erythromycin (96%), trimethoprim-sulfamethoxazole (79%) and tetracycline (77%) in a pediatric population (Cai et al., Infect Drug Resist 2018; 11:2461-2469) and (2) a survey of S. pneumoniae isolates from invasive infections in an older population (Intra et al., Front Public Health 2017; 5:169). A review publication, Kollef & Betthauser, Curr. Opin. Inf. Dis. 2019; 32:169-175, emphasizes increasing antibiotic resistance in common bacterial pathogens associated with community-acquired pneumonia (CAP), especially staphylococci and Streptococcus pneumonia.

Antimicrobial agents have long been used to treat microbial infections because of their therapeutic effects against microbial infections in humans and animals. Resistance to a previously therapeutically effective antimicrobial agent may be caused by a change in the infection-causing pathogen. Overuse and misuse of antimicrobials may be a factor in the growing problem of antimicrobial resistance, which continues to result in increasing types of pathogenic infections that are less responsive to previously effective antimicrobial agents. Antimicrobial resistance results in a lack of therapeutic options with which to treat pathogenic infections. Antimicrobial-resistant pathogens result in numerous deaths each year and are a serious worldwide public health challenge.

### SUMMARY OF THE INVENTION

The invention, in part, relates to compositions that can be used to synergistically treat a microbial infection. The compositions comprise one or more antimicrobial agents and a gelsolin agent. Methods of the invention, in part, relate to the administrations of such compositions to a subject, wherein the antimicrobial agent and the gelsolin agent act synergistically to treat a microbial infection in the subject.

According to an aspect of the invention, a composition is provided, the composition including a gelsolin agent and an antimicrobial agent in effective amounts to synergistically treat a microbial infection in a subject. In some embodiments, the antimicrobial agent is in a clinically acceptable amount and the administered gelsolin agent and antimicrobial agent synergistically enhance a therapeutic effect of administering the clinically acceptable amount of the antimicrobial agent and not the gelsolin agent to the subject. In certain embodiments, the clinically acceptable amount of the antimicrobial agent is an amount below a maximum tolerated dose (MTD) of the antimicrobial agent in the subject. In some embodiments, the MTD of the antimicrobial agent is a highest possible but still tolerable dose level of the antimicrobial agent for the subject. In some embodiments, the MTD of the antimicrobial agent is determined at least in part on a pre-selected clinical-limiting toxicity for the antimicrobial agent in the subject. In certain embodiments, the synergistically effective amount of the gelsolin agent and the antimicrobial agent decreases a minimum effective dose (MED) of the antimicrobial agent in the subject. In certain embodiments, the MED is a lowest dose level of the antimicrobial agent that provides a clinically significant response in average efficacy, wherein the response is statistically significantly greater than a response provided by a control that does not include the dose of the antimicrobial agent. In some embodiments, the synergistic therapeutic effect of the gelsolin agent and the antimicrobial agent includes increasing a likelihood of survival of the subject. In some embodiments, the synergistic therapeutic effect of the gelsolin agent and the antimicrobial agent includes reducing the microbial infection in the subject. In some embodiments, the microbial infection is a bacterial infection, and optionally is caused by a Pneumococcal species. In certain embodiments, the antimicrobial agent includes a β-lactam antibiotic. In some embodiments, the antimicrobial agent includes penicillin. In some embodiments, the microbial infection is caused by a type of Pseudomonas aeruginosa. In certain embodiments, the antimicrobial agent is an antimicrobial in the carbapenem class. In some embodiments, the antimicrobial agent is meropenem. In some embodiments, the antimicrobial agent includes an antifungal agent and the microbial infection includes a fungal infection. In certain embodiments, the antimicrobial agent includes an anti-parasitic agent and the microbial infection includes a parasitic infection. In certain embodiments, the antimicrobial agent comprises an antiviral agent and the microbial infection comprises a viral infection. In some embodiments, the subject is a mammal, optionally a human. In some embodiments, the gelsolin agent comprises plasma gelsolin (pGSN), and optionally is a recombinant pGSN. In some embodiments, the composition also includes a pharmaceutically acceptable carrier. In certain embodiments, the gelsolin agent comprises a gelsolin molecule, a functional fragment thereof, or a functional derivative of the gelsolin molecule. In some embodiments, the composition also includes a pharmaceutically acceptable carrier.

According to an aspect of the invention, a method of increasing a therapeutic effect of an antimicrobial agent on a microbial infection in a subject, the method comprising: administering to a subject having a microbial infection synergistically effective amounts of each of a gelsolin agent and an antimicrobial agent, wherein the administered gelsolin agent and antimicrobial agents have a synergistic therapeutic effect against the microbial infection in the subject, and the synergistic therapeutic effect is greater than a therapeutic effect of the antimicrobial agent administered without the gelsolin agent. In some embodiments, the antimicrobial agent is administered in a clinically acceptable amount. In some embodiments, the synergistic therapeutic effect against the microbial infection is greater than a control therapeutic effect against the microbial infection, wherein the control therapeutic effect is a sum of a therapeutic effect of the antimicrobial agent on the microbial infection plus a therapeutic effect of the gelsolin agent on the microbial infection when each of the antimicrobial agent and the gelsolin agent is administered without the other. In certain embodiments, the control therapeutic effect is equal to the individual therapeutic effect of the gelsolin agent. In some embodiments, the control therapeutic effect is equal to the individual therapeutic effect of the antimicrobial agent administered in a clinically acceptable amount. In certain embodiments, the synergistic therapeutic effect is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater than the control therapeutic effect. In some embodiments, the antimicrobial agent comprises an antibiotic agent and the microbial infection comprises a bacterial infection. In some embodiments, the antimicrobial agent comprises an antifungal agent and the microbial infection comprises a fungal infection. In certain embodiments, the antimicrobial agent comprises an anti-parasitic agent and the microbial infection comprises a parasitic infection. In some embodiments, the antimicrobial agent comprises an antiviral agent and the microbial infection comprises a viral infection. In some embodiments, the gelsolin agent comprises a gelsolin molecule, a functional fragment thereof, or a functional derivative of the gelsolin molecule. In some embodiments, the gelsolin molecule is a plasma gelsolin (pGSN). In certain embodiments, the gelsolin molecule is a recombinant gelsolin molecule. In some embodiments, the clinically acceptable amount of the antimicrobial agent is an amount below a maximum tolerated dose (MTD) of the antimicrobial agent. In some embodiments, the MTD of the antimicrobial agent is a highest possible but still tolerable dose level of the antimicrobial agent for the subject. In certain embodiments, the MTD of the antimicrobial agent is determined at least in part on a pre-selected clinically limiting toxicity for the antimicrobial agent. In some embodiments, the synergistically effective amount of the gelsolin agent and the antimicrobial agent decreases a minimum effective dose (MED) of the antimicrobial agent in the subject. In some embodiments, the synergistic therapeutic effect of the administration of the synergistically effective amount of each of the antimicrobial agent and the gelsolin agent reduces a level of the microbial infection in the subject compared to a control level of the microbial infection. In some embodiments, the control level of infection comprises a level of infection in the absence of administering the synergistically effective amount of each of the antimicrobial agent and the gelsolin agent. In certain embodiments, the level of the subject's microbial infection is at least 5%, 10%, 15%, 20%, 25%, 30%, 35%, 40%, 45%, 50%, 55%, 60%, 65%, 70%, 75%, 80%, 85%, 90%, 95%, or 100% lower than the control level of microbial infection. In some embodiments, the level of the microbial infection in the subject is determined, and a means of the determining comprises one or more of: an assay, observing the subject, assessing one or more physiological symptoms of the microbial infection in the subject, and assessing a likelihood of survival of the subject. In some embodiments, the physiological symptoms comprise one or more of: fever, malaise, and death. In certain embodiments, the physiological symptoms comprise lung pathology. In some embodiments, the physiological symptoms comprise weight loss. In some embodiments, the assay comprises a means for detecting the presence, absence, and/or level of a characteristic of the microbial infection in a biological sample from the subject. In some embodiments, the administration of the synergistically effective amount of each of the antimicrobial agent and the gelsolin agent increases the subject's likelihood of survival compared to a control likelihood of survival. In certain embodiments, the control likelihood of survival is a likelihood of survival in the absence of the administration of the synergistically effective amount of each of the antimicrobial agent and the gelsolin agent. In some embodiments, the increase in the subject's likelihood of survival is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% higher than the control likelihood of survival. In certain embodiments, the administration of the synergistically effective amount of each of the antimicrobial agent and the gelsolin agent reduces a level of lung pathology in the subject compared to a control level of lung pathology. In some embodiments, the control level of lung pathology is a level of lung pathology in the absence of the administration of the synergistically effective amount of each of the antimicrobial agent and the gelsolin agent. In certain embodiments, the level of lung pathology in the subject administered the synergistically effective amount of each of the antimicrobial agent and the gelsolin agent is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% lower than the control level of lung pathology. In some embodiments, the subject has a Pseudomonas aeruginosa bacterial infection. In some embodiments, the antimicrobial agent comprises carbapenem class, optionally meropenem. In certain embodiments, the bacterial infection is caused by a type of Streptococcus pneumoniae (pneumococcus). In some embodiments, the antimicrobial agent comprises a B-lactam antibiotic. In some embodiments, the antimicrobial agent comprises penicillin. In certain embodiments, the bacterial infection is caused by a type of Pseudomonas aeruginosa. In some embodiments, the antimicrobial agent is an antimicrobial in the carbapenem class. In some embodiments, the antimicrobial agent is meropenem. In certain embodiments, the bacterial infection is caused by one or more of: a gram-positive bacterium, a gram-negative bacterium, a tuberculosis bacillus, a non-tuberculous mycobacterium, a spirochete, an actinomycete, an Ureaplasma species bacterium, a Mycoplasma species bacterium, and a Chlamydia species bacterium. In some embodiments, the administration means of the gelsolin agent and the antimicrobial agent are independently selected from: oral, sublingual, buccal, intranasal, intravenous, intramuscular, intrathecal, intraperitoneal, subcutaneous, intradermal, topical, rectal, vaginal, intrasynovial, and intra-ocular administration. In some embodiments, the subject is a mammal, and optionally is a human. In certain embodiments, the gelsolin agent is a non-therapeutic gelsolin agent. In some embodiments, the antimicrobial agent is a non-therapeutic agent.

According to another aspect of the invention, a method for synergistically treating a microbial infection in a subject is provided, the method comprising, administering to a subject having a microbial infection an effective amount of each of a gelsolin agent and an antimicrobial agent wherein the administered gelsolin agent and antimicrobial agents have a synergistic therapeutic effect against the microbial infection in the subject compared to a control therapeutic effect, and the antimicrobial agent is administered in a clinically acceptable amount. In some embodiments, the control comprises a therapeutic effect of administering the clinically acceptable amount of the antimicrobial agent administered without administering the gelsolin agent. In certain embodiments, the clinically acceptable amount of the antimicrobial agent is an amount below a maximum tolerated dose (MTD) of the antimicrobial agent. In some embodiments, the MTD of the antimicrobial agent is a highest possible but still tolerable dose level of the antimicrobial agent for the subject. In some embodiments, the MTD of the antimicrobial agent is determined at least in part on a pre-selected clinical-limiting toxicity for the antimicrobial agent. In some embodiments, the synergistically effective amount of gelsolin agent and the antimicrobial agent decreases a minimum effective dose (MED) of the antimicrobial agent in the subject. In certain embodiments, the MED is a lowest dose level of the antimicrobial agent that provides a clinically significant response in average efficacy, wherein the response is statistically significantly greater than a response provided by a control that does not include the dose of the antimicrobial agent. In some embodiments, the synergistic therapeutic effect is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater than the control therapeutic effect. In some embodiments, the antimicrobial agent comprises an antibiotic agent and the microbial infection comprises a bacterial infection. In certain embodiments, the antimicrobial agent comprises an antifungal agent and the microbial infection comprises a fungal infection. In some embodiments, the antimicrobial agent comprises an anti-parasitic agent and the microbial infection comprises a parasitic infection. In some embodiments, the antimicrobial agent comprises an antiviral agent and the microbial infection comprises a viral infection. In certain embodiments, the gelsolin agent comprises a gelsolin molecule, a functional fragment thereof, or a functional derivative of the gelsolin molecule. In some embodiments, the gelsolin molecule is a plasma gelsolin (pGSN). In some embodiments, the gelsolin molecule is a recombinant gelsolin molecule. In certain embodiments, the synergistic therapeutic effect of the administration of the synergistically effective amount of each of the antimicrobial agent and the gelsolin agent reduces a level of the microbial infection in the subject compared to a control level of the microbial infection. In some embodiments, the control level of infection comprises a level of infection in the absence of administering the synergistically effective amount of each of the antimicrobial agent and the gelsolin agent. In certain embodiments, the level of the subject's microbial infection is at least 5%, 10%, 15%, 20%, 25%, 30%, 35%, 40%, 45%, 50%, 55%, 60%, 65%, 70%, 75%, 80%, 85%, 90%, 95%, or 100% lower than the control level of microbial infection. In some embodiments, the level of the microbial infection in the subject is determined, and a means of the determining comprises one or more of: an assay, observing the subject, assessing one or more physiological symptoms of the microbial infection in the subject, and assessing a likelihood of survival of the subject. In certain embodiments, the physiological symptoms comprise one or more of: fever, malaise, and death. In some embodiments, the physiological symptoms comprise weight loss. In some embodiments, the physiological symptoms comprise lung pathology. In certain embodiments, the assay comprises a means for detecting the presence, absence, and/or level of a characteristic of the microbial infection in a biological sample from the subject. In some embodiments, the administration of the synergistically effective amount of each of the antimicrobial agent and the gelsolin agent increases the subject's likelihood of survival compared to a control likelihood of survival. In certain embodiments, the control likelihood of survival is a likelihood of survival in the absence of the administration of the synergistically effective amount of each of the antimicrobial agent and the gelsolin agent. In some embodiments, the increase in the subject's likelihood of survival is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% higher than the control likelihood of survival. In some embodiments, the administration of the synergistically effective amount of each of the antimicrobial agent and the gelsolin agent reduces a level of lung pathology in the subject compared to a control level of lung pathology. In certain embodiments, the control level of lung pathology is a level of lung pathology in the absence of the administration of the synergistically effective amount of each of the antimicrobial agent and the gelsolin agent. In some embodiments, the level of lung pathology in the subject administered the synergistically effective amount of each of the antimicrobial agent and the gelsolin agent is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% lower than the control level of lung pathology. In some embodiments, the subject has a Pseudomonas aeruginosa bacterial infection. In certain embodiments, the antimicrobial agent comprises carbapenem class, optionally meropenem. In some embodiments, the bacterial infection is caused by a type of Streptococcus pneumoniae (pneumococcus). In certain embodiments, the antimicrobial agent comprises a ß-lactam antibiotic. In some embodiments, the antimicrobial agent comprises penicillin. In some embodiments, the bacterial infection is caused by one or more of: a gram-positive bacterium, a gram-negative bacterium, a tuberculosis bacillus, a non-tuberculous mycobacterium, a spirochete, an actinomycete, an Ureaplasma species bacterium, a Mycoplasma species bacterium, and a Chlamydia species bacterium. In certain embodiments, the administration means of the gelsolin agent and the antimicrobial agent are independently selected from: oral, sublingual, buccal, intranasal, intravenous, intramuscular, intrathecal, intraperitoneal, subcutaneous, intradermal, topical, rectal, vaginal, intrasynovial, and intra-ocular administration. In some embodiments, the subject is a mammal. In some embodiments, the gelsolin agent is a non-therapeutic gelsolin agent. In certain embodiments, the antimicrobial agent is a non-therapeutic agent.

According to another aspect of the invention, a pharmaceutical composition comprising an antimicrobial agent and a gelsolin agent that synergistically increase a therapeutic effect of the antimicrobial agent on a microbial infection for use in a method of treatment of a subject, wherein: the subject has a microbial infection, the method comprising: administering the pharmaceutical composition comprising synergistically effective amounts of each of the gelsolin agent and the antimicrobial agent in an amount effective to treat the microbial infection in the subject, wherein the synergistic therapeutic effect is greater than a therapeutic effect of the antimicrobial agent administered without the gelsolin agent. In some embodiments, the gelsolin agent and the antimicrobial agent are administered to a subject separately or simultaneously. In certain embodiments, the antimicrobial agent is administered in a clinically acceptable amount and the administered gelsolin agent and antimicrobial agent synergistically enhance a therapeutic effect of administering the clinically acceptable amount of the antimicrobial agent and not the gelsolin agent to the subject. In some embodiments, the clinically acceptable amount of the antimicrobial agent is an amount below a maximum tolerated dose (MTD) of the antimicrobial agent in the subject. In some embodiments, the MTD of the antimicrobial agent is a highest possible but still tolerable dose level of the antimicrobial agent for the subject. In certain embodiments, the MTD of the antimicrobial agent is determined at least in part on a pre-selected clinical-limiting toxicity for the antimicrobial agent in the subject. In some embodiments, the synergistically effective amount of the gelsolin agent and the antimicrobial agent decreases a minimum effective dose (MED) of the antimicrobial agent in the subject. In some embodiments, the MED is a lowest dose level of the antimicrobial agent that provides a clinically significant response in average efficacy, wherein the response is statistically significantly greater than a response provided by a control that does not include the dose of the antimicrobial agent. In certain embodiments, the synergistic therapeutic effect of the gelsolin agent and the antimicrobial agent comprises increasing a likelihood of survival of the subject. In some embodiments, the synergistic therapeutic effect of the gelsolin agent and the antimicrobial agent comprises reducing the microbial infection in the subject. In some embodiments, the microbial infection is a bacterial infection, and optionally is caused by a Pneumococcal species. In certain embodiments, the antimicrobial agent comprises penicillin. In some embodiments, the bacterial infection is caused by a type of Pseudomonas aeruginosa. In some embodiments, the antimicrobial agent is an antimicrobial in the carbapenem class. In certain embodiments, the antimicrobial agent is meropenem. In some embodiments, the antimicrobial agent comprises an antifungal agent and the microbial infection comprises a fungal infection. In certain embodiments, the antimicrobial agent comprises an anti-parasitic agent and the microbial infection comprises a parasitic infection. In some embodiments, the antimicrobial agent comprises an antiviral agent and the microbial infection comprises a viral infection. In some embodiments, the subject is a mammal. In certain embodiments, the gelsolin agent comprises plasma gelsolin (pGSN), and optionally is a recombinant pGSN. In some embodiments, wherein the pharmaceutical composition also includes a pharmaceutically acceptable carrier. In some embodiments, the gelsolin agent comprises a gelsolin molecule, a functional fragment thereof, or a functional derivative of the gelsolin molecule. In certain embodiments, the pharmaceutical composition also includes a pharmaceutically acceptable carrier.

In yet another aspect of the invention, a method for treating a viral infection in a subject is provided, the method including administering to a subject having a viral infection an effective amount of a gelsolin agent, wherein the gelsolin agent is administered at least 3, 4, 5, 6, 7, 8, 9, or more days after infection of the subject with the viral infection, and is not administered the day the subject is infected with the viral infection, 1 day after the subject is infected with the viral infection, or 2 days after the subject is infected with the viral infection. In some embodiments, the effective amount of the gelsolin agent has an increased therapeutic effect against the viral infection in the subject, compared to a control therapeutic effect. In certain embodiments, the control therapeutic effect comprises a therapeutic effect of when gelsolin agent is not administered to the subject. In certain embodiments, the antiviral agent comprises one or more of: oseltamivir phosphate, zanamivir, peramivir, and baloxavir marboxil. In some embodiments, the therapeutic effect of the administered gelsolin agent is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater than the control therapeutic effect. In some embodiments, the gelsolin agent comprises a gelsolin molecule, a functional fragment thereof, or a functional derivative of the gelsolin molecule. In certain embodiments, the gelsolin molecule is a plasma gelsolin (pGSN). In some embodiments, the gelsolin molecule is a recombinant gelsolin molecule. In some embodiments, the therapeutic effect of the administration of the gelsolin agent reduces a level of the viral infection in the subject compared to a control level of the viral infection, wherein the control level of infection comprises a level of infection in the absence of administering the gelsolin agent. In certain embodiments, the level of the subject's viral infection is at least 5%, 10%, 15%, 20%, 25%, 30%, 35%, 40%, 45%, 50%, 55%, 60%, 65%, 70%, 75%, 80%, 85%, 90%, 95%, or 100% lower than the control level of viral infection. In some embodiments, the level of the viral infection in the subject is determined, and a means of the determining comprises one or more of: an assay, observing the subject, assessing one or more physiological symptoms of the viral infection in the subject, and assessing a likelihood of survival of the subject. In some embodiments, the physiological symptoms comprise one or more of: fever, malaise, weight loss, and death. In some embodiments, the assay comprises a means for detecting the presence, absence, and/or level of a characteristic of the viral infection in a biological sample from the subject. In certain embodiments, the administration of the effective amount of the gelsolin agent increases the subject's likelihood of survival compared to a control likelihood of survival. In some embodiments, the control likelihood of survival is a likelihood of survival in the absence of the administration of the gelsolin agent. In certain embodiments, the increase in the subject's likelihood of survival is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% higher than the control likelihood of survival. In some embodiments, the administration means of the gelsolin agent is selected from: oral, sublingual, buccal, intranasal, intravenous, inhalation, intramuscular, intrathecal, intraperitoneal, subcutaneous, intradermal, topical, rectal, vaginal, intrasynovial, and intra-ocular administration. In some embodiments, the subject is a mammal, and optionally is a human. In certain embodiments, the method also includes treating the subject with an antiviral agent on one or more days prior to the administration of the gelsolin agent to the subject, wherein the antiviral agent is administered on one or more of: the day the subject is infected with the viral infection, one day after the subject is infected with the viral infection, and two days after the subject is infected with the viral infection. In some embodiments, a synergistically effective amount of each of a gelsolin agent and the antiviral agent are administered to the subject and have a synergistic therapeutic effect against the viral infection, compared to a control therapeutic effect, and the antiviral agent is administered in a clinically acceptable amount. In some embodiments, the control comprises a therapeutic effect of administering the clinically acceptable amount of the antiviral agent administered without administering the gelsolin agent. In certain embodiments, the clinically acceptable amount of the antiviral agent is an amount below a maximum tolerated dose (MTD) of the antiviral agent. In some embodiments, the MTD of the antiviral agent is a highest possible but still tolerable dose level of the antiviral agent for the subject. In some embodiments, the MTD of the antiviral agent is determined at least in part on a pre-selected clinical-limiting toxicity for the antiviral agent. In certain embodiments, the synergistically effective amount of gelsolin agent and the antiviral agent decreases a minimum effective dose (MED) of the antiviral agent in the subject. In some embodiments, the MED is a lowest dose level of the antiviral agent that provides a clinically significant response in average efficacy, wherein the response is statistically significantly greater than a response provided by a control that does not include the dose of the antiviral agent. In certain embodiments, the administration means of the gelsolin agent and the antiviral agent are independently selected from: oral, sublingual, buccal, intranasal, inhalation, intravenous, intramuscular, intrathecal, intraperitoneal, subcutaneous, intradermal, topical, rectal, vaginal, intrasynovial, and intra-ocular administration.

### BRIEF DESCRIPTION OF THE DRAWINGS

FIG. 1A-F shows graphs of results of systemic experiments that measure improvements in host defense against bacterial pneumonia following administration of pGSN. In vitro, pGSN improves macrophage uptake (FIG. 1A) and killing of internalized pneumococci (FIG. 1B) when present at 125-250 μg/ml, similar to normal plasma levels. In vivo, pGSN (10 mg s.c. 2h before and 8 and 20 h after infection improved bacterial clearance (fewer surviving bacteria at 24 h) in B16 mice challenged with 10⁵ pneumococci by i.n. insufflation (FIG. 1C); similar results were seen when pGSN was administered as an aerosol for 15 or 30 minutes prior to infection (FIG. 1D). Systemic pGSN (s.c.) improved survival in primary (FIG. 1E, using 3×10⁵ CFU inoculum) or secondary post-influenza pneumococcal pneumonia (FIG. 1F, using 500 CFU inoculum on day 7 after mild influenza infection with PR8) even in the absence of any antibiotic treatment. *=p<0.05 vs control, n=6-12 per group. All experiments used serotype 3 Strep. Pneumoniae.

FIG. 2A-B provides graphs showing that the effect of pGSN on macrophages requires NOS3. FIG. 2A shows experimental results that demonstrated macrophage killing of pneumococci in vitro. FIG. 2B shows experimental results indicating macrophage clearance of bacteria in vivo. Macrophage clearance of bacteria was no longer enhanced if NOS3-deficient cells or animals were used. *=p<0.01.

FIG. 3A-B provides graphs of studies of antibiotic-sensitive pneumococcal pneumonia. FIG. 3A shows results of treatment with pGSN (5 mg i.p. on days 2 and 3 after infection) demonstrating improved survival in mice infected with serotype 3 pneumococci (*=p=0.01, n=20/group, summary of 2 trials, 10 mice per group per trial). FIG. 3B provides results of treatment with penicillin (PEN, 100 μg i.m. on days 2 and 3 after infection) indicating improved survival in mice infected with serotype 3 pneumococci (*=p=0.02, n=8-9/group, single trial).

FIG. 4A-C provides graphs of results of studies with antibiotic-resistant pneumococcal pneumonia. Results in FIG. 4A demonstrate treatment with pGSN (5 mg i.p. daily starting on day 1 after infection) improved survival in mice infected with serotype 14 pneumococci compared to vehicle or penicillin (PEN, 1 mg dose i.m daily) (*, p=0.02, 0.04 respectively, log rank comparisons after Sidak adjustment for multiple comparisons). Combined treatment with pGSN and penicillin also resulted in higher survival compared to vehicle or penicillin (**, p=0.0001 for both comparisons, log rank with Sidak adjustment for multiple comparisons). Survival of the pGSN vs. pGSN+PEN groups was not statistically significant after Sidak adjustment for multiple comparisons (p=47 n=38-41/group, summary of 4 trials, 8-11 mice per group per trial). Assessment of (FIG. 4B) weight loss and (FIG. 4C) morbidity showed more rapid recovery of weight and a lower morbidity index in the pGSN or pGSN+PEN groups (mean values for each day shown, p=0.001, p=0.04 respectively, ANOVA; n=38-41 per group in 4 trials for B, n=30 per group in 3 trials for C; the last observation for any mouse was carried forward after death).

FIG. 5 provides a table with data results from nine experiments in which testing delayed administration of four treatments was assessed. FIG. 5 presents details of nine experiments, including pilot and range-finding trials. Column H shows a change in bacterial growth method obtained using a method for 2X growth in BHI broth for penicillin-resistant pneumococci [Restrepo AV et al., BMC Microbiol 2005; 5:34] for superior growth results. The data provided in the table demonstrate that in all nine experiments, survival was highest in the PEN+pGSN group. Survival in the PEN+pGSN group was higher than in the pGSN group, and survival in both were higher than in the vehicle or PEN alone groups. The survival differences were statistically significant, as determined by analysis of all the nine studies pooled using log rank analysis along with Sidak correction for multiple comparisons. Details of the results of statistical analysis of the final four experiments (#6-9) are summarized in FIG. 4A-C.

FIG. 6 provides a table with data from three experiments assessing survival following administration of meropenem doses with or without rhu-pGSN to neutropenic mice. FIG. 6 presents details of three experiments in which meropenem doses as indicated were administered subcutaneously beginning at 3 h post-infection with MDR P. aeruginosa and q8h thereafter for 5 days. Meropenem doses were administered either with or without rhu-pGSN. rhu-pGSN was administered as 12 mg via intraperitoneal injection on Days −1, 0, 1, 2, 3, 4, and 5. n/N=number of surviving mice/Number of treated mice.

FIG. 7A-C provides graphs demonstrating the survival benefit observed with combined meropenem and rhu-pGSN treatment. BALB/c-mice made neutropenic with cyclophosphamide (BALB/c-Cy mice) were infected with the UNC-D strain of P. aeruginosa and treated with either meropenem alone (1250 mg/kg/day subcutaneously q8h for 5 days beginning 3 h post-infection) or in combination with pGSN (12 mg/day intraperitoneally daily for days −1 to +5). Mice were euthanized upon reaching endpoint criteria or at the study conclusion on Day 7. Survival analysis was conducted by log-rank test using two studies of n=8 group size (FIG. 7A and FIG. 7B), where the control mortality rate at Day 7 was ≥50% with the same 1250 mg meropenem dose. The results were then analyzed by combining these two separate studies (FIG. 7C). The p values refer to the survival advantage of combination therapy over meropenem alone. MTD, mean time to death.

FIG. 8A-C provides graphs illustrating that rhu-pGSN administration reduces bacterial counts in the lungs. Two studies were performed in which BALB/c-Cy mice were infected with the UNC-D strain of P. aeruginosa and treated with either meropenem alone (1250 mg/kg/day subcutaneously q8h for 5 days beginning 3 h post-infection) or in combination with pGSN (12 mg/day intraperitoneally daily for days −1 to +5). Mice were euthanized upon reaching endpoint criteria (open circle) or survivors at the study conclusion on Day 7 (closed circle). Bacteria were enumerated from homogenized lung by plate count. FIG. 8A shows a graph of the results of the first study; FIG. 8B shows a graph of the results from the second study. Individual and combined data were analyzed for the two studies and with pairwise analysis of meropenem therapy alone (Mero) versus in combination with pGSN. p values refer to unpaired Student t-test comparisons of combination therapy versus meropenem alone. The lines at the bottom of the graph indicate the limit of detection. FIG. 8C shows a graph of combined data from the two studies shown in FIG. 8A and FIG. 8B.

FIG. 9A-C provides graphs illustrating that rhu-pGSN limits infection-induced lung injury. Two studies were performed in which BALB/c-Cy mice were infected with the UNC-D strain of P. aeruginosa and treated with either meropenem alone (1250 mg/kg/day subcutaneously q8h for 5 days beginning 3 h post-infection) or in combination with pGSN (12 mg/day intraperitoneally daily for days −1 to +5). Mice were euthanized upon reaching endpoint criteria (open circle) or survivors at the study conclusion on Day 7 (closed circle). A representative section of lung was excised from the lung and processed for H&E staining and scoring. FIG. 9A shows a graph of the results of the first study; FIG. 9B shows a graph of the results of the second study. Data was analyzed for the two individual studies separately and combined with pairwise analysis of meropenem therapy alone (Mero) or in combination with pGSN. The p values refer to unpaired Student t-test comparisons of combination therapy versus meropenem alone. FIG. 9C shows a graph of combined data from the two studies shown in FIG. 9A and FIG. 9B.

FIG. 10 provides a table with data of overall survival with minor lung injury from surviving mice treated with different doses of meropenem. Meropenem doses as indicated were administered subcutaneously beginning at 3 h post-infection and q8h thereafter for 5 days. rhu-pGSN was administered as 12 mg via intraperitoneal injection on Days −1, 0, 1, 2, 3, 4, and 5. Asterisk (*) indicates that a total of 3 mice (all in experiment #2) were euthanized at 20 hours post-challenge but had no lung injury; there was one mouse in each of the three meropenem+rhu-pGSN treatment groups. Excluding these 3 mice from the rhu-pGSN tallies yielded a final count of 41/61 (67.2%). n/N=number of surviving mice with composite Lung Injury Scores ≤2/Number of treated mice.

FIG. 11A-D presents graphs from two experiments demonstrating restoration of baseline temperature in mice treated with either meropenem alone or with meropenem plus rhu-pGSN. In both experiments, BALB/c-Cy mice were infected with the UNC-D strain of P. aeruginosa. FIG. 11A shows a graph of temperatures from mice in the first experiment treated with meropenem alone (1250 mg/kg/day subcutaneously q8h for 5 days beginning 3 h post-infection); FIG. 11C shows a graph of temperatures from mice in the second experiment treated with meropenem alone (same regimen as in FIG. 11A). FIG. 11B shows a graph of temperatures from mice in the first experiment treated with meropenem in combination with rhu-pGSN (12 mg/day intraperitoneally daily for days −1 to +5); FIG. 11D shows a graph of temperatures from mice in the second experiment treated with meropenem in combination with rhu-pGSN (same regimen as in FIG. 11B). Animal temperatures were monitored every 8 hours post-infection until the end of study. Mice were euthanized upon reaching endpoint criteria (open circles) or at the study conclusion on Day 7 (closed circles).

FIG. 12 provides a table showing details of treatment trials using recombinant human plasma gelsolin (rhu-pGSN) in murine influenza. *=Treatment benefit scored as Yes if % survival ≥10% better with pGSN vs. Vehicle; No if % survival <10% better with pGSN.

FIG. 13 provides a summary of survival data using different treatment regimens. pGSN is plasma gelsolin.

FIG. 14A-H provides results of survival and morbidity analysis of different treatment regimens. Comparison of survival rates (FIG. 14A, C, E, & G) and morbidity (FIG. 14B, D, F, & H) in mice treated with rhu-pGSN or vehicle. (FIG. 14A-B) Results for all 18 trials (typically 10 or more mice per group, see details in FIGS. 12 and 13) using delayed treatment. Some trials initiated treatment in different arms on day 6 or day 3. (FIG. 14C-D) Results for 13 trials using delayed treatment starting on day 6 or later. (FIG. 14E-F) Results for eight trials using treatment starting on day 3. (FIG. 14G-H) Results for four trials starting with an initially lower dose on day 3 with an increased dose starting on day 6/7. *=0.000001, 0.00001, 0.0005, 0.0005 for FIG. 14A, C, E, & G, respectively; p<0.0001 for FIG. 14B, D, F, & H.

FIG. 15 provides experimental results showing the top 50 up-and down-regulated differentially expressed genes in lung tissue from vehicle or rhu-pGSN treated animals (Day 9). Heat map showing top 50 down-regulated (left) and up-regulated (right) genes in the lungs of rhu-pGSN treated animals on day 9 (range −2 to +2, shown in scale on right).

FIG. 16 shows top 10 down-regulated Gene Ontology (GO) processes and pathways in plasma gelsolin (pGSN)-treated lung tissue (Day 9).

### DETAILED DESCRIPTION

The present invention is based, in part, on the discovery that administering a gelsolin agent and an antimicrobial agent to a subject with a microbial infection can result in a synergistic therapeutic effect of the two agents that reduces the microbial infection. The invention includes, in some aspects, a therapeutic composition comprising an exogenous gelsolin agent and an antimicrobial agent that when administered to a subject with a microbial infection act synergistically in the subject and their synergistic action results in a therapeutic effect that is greater than the therapeutic effect of administering to the subject a clinically acceptable dose of either the gelsolin agent or the antimicrobial agent, in the absence of administering the other to the subject. Certain methods of the invention include administering a pharmaceutical composition of the invention to a subject with a microbial infection, in an amount that is effective to produce a synergistic therapeutic effect against the microbial infection in the subject. Some methods of the invention include delayed-dose administration of a gelsolin agent to a subject with a viral infection, which enhances treatment of the viral infection in the subject.

### Synergistic Therapeutic Effects

Methods of the invention include producing a synergistic therapeutic effect in a subject with a microbial infection to reduce and treat the microbial infection. It has been determined that even if one or both of a gelsolin agent and a antimicrobial agent has no statistically significant individual therapeutic effect against a microbial infection, they can be administered in conjunction with each other and produce a synergistic therapeutic effect against the microbial infection. Thus, in some aspects of the invention, a microbial infection in a subject that is caused by a microbe resistant to one or more antimicrobial agents can be effectively treated using a synergistic therapeutic method of the invention because of the newly discovered synergistic therapeutic effect of administering synergistically effective amounts of a gelsolin agent and an antimicrobial agent to a subject.

The term “individual therapeutic effect” as used herein in reference to an agent such as a gelsolin agent or an antimicrobial agent means a therapeutic effect of the agent when it is administered to a subject having a microbial infection. With respect to methods and compositions of the invention, an individual therapeutic effect of a gelsolin agent is a therapeutic effect against a microbial infection in a subject that results from administering the gelsolin agent to the subject in the absence of administering an antimicrobial agent to the subject. In reference to methods and compositions of the invention an individual therapeutic effect of an antimicrobial agent is a therapeutic effect against a microbial infection in a subject that results from administering the antimicrobial agent to the subject in the absence of administering a gelsolin agent.

As is understood in the art, a synergistic therapeutic effect is a therapeutic effect resulting from interaction between two or more drugs that causes a total therapeutic effect of the drugs to be greater than the sum of the individual therapeutic effects of each drug. With respect to methods of the invention, a total therapeutic effect of administered gelsolin and antimicrobial agents is greater than the sum of the individual therapeutic effect of the gelsolin agent plus the individual therapeutic effect of the antimicrobial agent. In a non-limiting example, a subject with a Streptococcus pneumonia infection may be treated with a method of the invention that includes administering synergistically effective amounts of a plasma gelsolin (pGSN) agent and penicillin to the subject, to result in a synergistic therapeutic effect against the infection in the subject. In this example, the therapeutic effect of administering both the pGSN agent and the penicillin is greater than the sum of the individual therapeutic effect of the amount of the pGSN plus the individual therapeutic effect of the amount of the penicillin on the Streptococcus pneumonia infection.

In some embodiments, a method of the invention includes administering a synergistically effective amount of each of a gelsolin agent and a non-therapeutic antimicrobial agent to a subject with a microbial infection. The synergistic effect of the combined administration may increase the therapeutic effect of the antimicrobial agent. The term “non-therapeutic agent” is used herein in reference to an antimicrobial agent that does not have a statistically significant individual therapeutic effect against a microbial infection in a subject. It should be understood that a non-therapeutic agent as used with respect to methods and compositions of the invention is not an antimicrobial agent referred to in the art as a “therapeutic agent” or an “antimicrobial therapeutic agent.” For example, to a health care practitioner an antimicrobial agent without a statistically significant individual therapeutic effect against a microbial infection when administered in a clinically acceptable amount, would not be designated a therapeutic agent to administer to a subject having that microbial infection. Similarly, it has been recognized in the art that penicillin does not have a statistically significant individual therapeutic effect against certain microbial infections, and as such penicillin would be understood to be and defined as a “non-therapeutic agent” with respect to those infections. In certain embodiments of the invention an antimicrobial agent is a non-therapeutic agent with respect to its individual therapeutic effect against a microbial infection in a subject. In some embodiments of the invention an antimicrobial agent is a non-therapeutic agent with respect to its individual therapeutic effect against an antimicrobial-resistant microbial infection in a subject. A gelsolin agent that lacks a statistically significant individual therapeutic effect against a microbial infection in a subject may be referred to herein as a non-therapeutic agent with respect to the microbial infection.

### Individual and Synergistic Therapeutic Effects

Certain embodiments of methods and compositions of the invention include one or more agents that lack an individual therapeutic effect against the microbial infection in a subject. In some instances a gelsolin agent may have an individual therapeutic effect against a microbial infection and an antimicrobial agent may not have a statistically significant individual therapeutic effect. In the case of antimicrobials, a lack of an individual therapeutic effect of an antimicrobial agent against a microbial infection, may or may not be due to antimicrobial resistance in a microbe that causes the microbial infection. The term “resistant” used herein in relation to a microbe or a microbial infection means a microbe that is not killed or reduced, respectively, by the antimicrobial agent. In some embodiments of the invention, an individual therapeutic effect of an antimicrobial agent on an antimicrobial resistant microbe or infection may be zero.

In certain circumstances, a microbial infection in a subject results from a microbe that is resistant to an individual therapeutic effect of an antimicrobial agent. Acquired antimicrobial resistance may be understood as an ability of a disease-causing microbe to survive exposure to an antimicrobial agent that was previously an effective treatment of the disease. A microbe that is “antimicrobial resistant” may be the cause of a microbial infection in a subject and one or more antimicrobials are ineffective against the microbial infection, including one or more that were previously known to be therapeutically effective against the microbial infection. In a non-limiting example, a pneumococcal infection in a subject may result from the presence in the subject of a Streptococcus pneumonia bacterium that is resistant to a therapeutic effect of one or more antibiotic agents.

It will be understood that in certain embodiments of the invention the amount of a gelsolin agent administered and the amount of an antimicrobial agent administered are each clinically acceptable amounts for administration to the subject. It is known that certain microbial infections are not reduced by administration of an antimicrobial infection administered in a clinically acceptable amount. For example, in certain instances the microbe that causes the microbial infection is resistant to the administered antimicrobial agent, and in other such instances the microbe that causes the microbial infection is not sufficiently killed by the administration of a clinically acceptable amount of the antimicrobial agent. Although in either circumstance it may be possible to administer the antimicrobial agent in an amount sufficient to reduce the microbial infection in a subject, the amount required is a clinically unacceptable amount because it results in toxicity and/or other detrimental physiological effects in the subject. In contrast, the synergistic therapeutic effects of certain embodiments of methods of the invention permit administration of clinically acceptable amount of an antimicrobial agent that successfully reduces a microbial infection in a subject with statistically significantly less toxicity and fewer detrimental side effects in the subject.

In some embodiments of the invention, a clinically acceptable amount of the antimicrobial agent is an amount below a maximum tolerated dose (MTD) of the antimicrobial agent. It is understood in the art how a MTD can be determined for an individual in order to prevent or reduce negative side effects of administering a pharmacological agents. In some embodiments of the invention, an MTD of an antimicrobial agent is a highest possible dose level of the antimicrobial agent for the subject that is a dose that is tolerable to the subject. A tolerable dose may be determined based on side effects at a given dose level, including but not limited to: subject discomfort, physiological distress, increased risk of subject death, etc. In some embodiments of the invention, an MTD of an antimicrobial agent administered to a subject is determined at least in part based on a pre- selected clinically limiting toxicity for the antimicrobial agent. For example, a dose or amount of an antimicrobial that is effective to reduce or kill a microbe resistant to that antimicrobial agent may when administered to a subject, result in clinically unacceptable toxicity and/or detrimental side effects in the subject.

Methods of the invention are advantageous in that they can be used with lower doses of antimicrobial agents because of the synergistic therapeutic effects of administering both the antimicrobial agent and gelsolin agent to a subject with a microbial infection. In some embodiments of methods of the invention synergistically effective amounts of the gelsolin agent and the antimicrobial agent decreases a minimum effective dose (MED) of the antimicrobial agent in the subject. It should be understood that an amount or dose of a gelsolin agent and an amount or dose of an antimicrobial agent are independently selected, clinically acceptable amounts and doses.

In some instances an amount of a gelsolin agent and/or an amount of an antimicrobial agent does not have an individual therapeutic effect against a microbial infection in a subject. In some instances an amount of a gelsolin agent and/or an amount of an antimicrobial agent has an individual therapeutic effect against a microbial infection that is greater than zero. Table 1 illustrates relationships between independent therapeutic effects resulting from an amount of a gelsolin agent administered to a subject with a microbial infection, independent therapeutic effects resulting from an amount of an antimicrobial agent administered to a subject with a microbial infection, and synergistic therapeutic effects resulting from the amount of the gelsolin agent and the amount of the antimicrobial agent administered to a subject with the microbial infection. In each situation shown, the synergistic therapeutic effect is greater than the sum of the independent therapeutic effect of each of the gelsolin agent and the antimicrobial agent.

TABLE 1
Independent and synergistic therapeutic effects of selected amount
of Gelsolin Agent and selected amount of antimicrobial agent.

| | | Gelsolin Agent |
|---|---|---|
| Gelsolin | Antimicrobial | with Antimicrobial |
| Agent Independent | Agent Independent | Agent Synergistic |
| Therapeutic Effect | Therapeutic Effect | Therapeutic Effect |
| 0 | 0 | >0 |
| X, where X > 0 | 0 | >X |
| 0 | Y, where Y is >0 | >Y |
| X, where X > 0 | Y, where Y >0 | >X + Y |

### Therapeutic Compositions and Methods

A synergistic therapeutic effect of composition of the invention or a treatment method of the invention, (also referred to herein as a “response” to a treatment method of the invention) can be determined, for example, by detecting one or more physiological effects of the treatment, such as the decrease or lack of symptoms following administration of the synergistic treatment. Additional means of monitoring and assessing a microbial infection in a subject, determining one or more of presence, absence, level, severity, change in severity, etc. of microbial infections in subjects in response to treatment are well known in the art and may be utilized in conjunction with some embodiments of methods set forth herein.

Methods of the invention include administering a synergistic combination of a gelsolin agent and an antimicrobial agent to a subject with a microbial infection, each in an amount effective to result in a synergistic therapeutic effect to reduce the microbial infection in the subject. The gelsolin agent and the antimicrobial agent can be administered simultaneously. The gelsolin agent and the antimicrobial agent can be administered in the same or separate formulations, but are administered to be in the subject at the same time.

Methods and compositions of the invention may be used to treat a microbial infection. As used herein, the terms “treat”, “treated”, or “treating” when used in relation to a microbial infection may refer to a prophylactic treatment that decreases the likelihood of a subject developing the microbial infection, and may be used to refer to a treatment after a subject has developed a microbial infection in order to eliminate or ameliorate the microbial infection, prevent the microbial infection from becoming more advanced or severe, and/or to slow the progression of the microbial infection compared to the progression of the microbial infection in the absence of a therapeutic method of the invention.

### Gelsolin Agents

Gelsolin is a highly conserved, multifunctional protein, initially described in the cytosol of macrophages and subsequently identified in many vertebrate cells (Piktel E. et al., Int J Mol Sci 2018; 19: E2516; Silacci P. et al., Cell Mol Life Sci 2004; 61:2614-23.) A unique property of gelsolin is that its gene expresses a splice variant coding for a distinct plasma isoform (pGSN), which is secreted into extracellular fluids and differs from its cytoplasmic counterpart (cGSN) by expressing an additional sequence of 25 amino acids. pGSN normally circulates in mammalian blood at concentrations of 200-300 μg/ml, placing it among the most abundant plasma proteins. The term “gelsolin agent” as used herein means a composition that includes a gelsolin molecule, a functional fragment thereof, or a functional derivative of the gelsolin molecule. In some embodiments of the invention, a gelsolin agent only includes one or more of the gelsolin molecule, a functional fragment thereof, or a functional derivative of the gelsolin molecule. In certain embodiments of the invention a gelsolin agent may include one of more additional components, non-limiting examples of which are detectable labels, carriers, delivery agents, etc. In certain aspects of the invention a gelsolin molecule is a plasma gelsolin (pGSN) and in certain instances, a gelsolin molecule is a cytoplasmic GSN. A gelsolin molecule included in compositions and methods of the invention may be a recombinant gelsolin molecule.

As used herein, the term “gelsolin agent” is a compound that includes an exogenous gelsolin molecule. The term “exogenous” as used herein in reference to a gelsolin molecule means a gelsolin molecule administered to a subject, even if the same gelsolin molecule is naturally present in the subject, which may be referred to as an endogenous gelsolin molecule. A gelsolin agent included in a method or composition of the invention may be a wild-type gelsolin molecule (GenBank accession No.: X04412), isoforms, analogs, variants, fragments or functional derivatives of a gelsolin molecule.

In some embodiments of the invention may include a “gelsolin analog,” which as used herein refers to a compound substantially similar in function to either the native gelsolin or to a fragment thereof. Gelsolin analogs include biologically active amino acid sequences substantially similar to the gelsolin sequences and may have substituted, deleted, elongated, replaced, or otherwise modified sequences that possess bioactivity substantially similar to that of gelsolin. For example, an analog of gelsolin is one which does not have the same amino acid sequence as gelsolin but which is sufficiently homologous to gelsolin so as to retain the bioactivity of gelsolin. Bioactivity can be determined, for example, by determining the properties of the gelsolin analog and/or by determining the ability of the gelsolin analog to reduce or prevent the effects of an infection. Gelsolin bioactivity assays known to those of ordinary skill in the art.

Certain embodiments of methods and compositions of the invention include fragments of a gelsolin molecule. The term “fragment” is meant to include any portion of a gelsolin molecule which provides a segment of gelsolin that maintains at least a portion or substantially all of a level of bioactivity of the “parent” gelsolin; the term is meant to include gelsolin fragments made from any source, such as, for example, from naturally-occurring peptide sequences, synthetic or chemically-synthesized peptide sequences, and genetically engineered peptide sequences. The term “parent” as used herein in reference to a gelsolin fragment or derivative molecule means the gelsolin molecule from which the sequence of the fragment or derivative originated.

In certain embodiments of methods and compositions of the invention, a gelsolin fragment is a functional fragment and retains at least some up to all of the function of its parent gelsolin molecule. Methods and compositions of the invention, may in some embodiments include a “variant” of gelsolin. As used herein a gelsolin variant may be a compound substantially similar in structure and bioactivity either to native gelsolin, or to a fragment thereof. In certain aspects of the invention, a gelsolin variant is referred to as a functional variant, and retains at least some up to all of the function of its parent gelsolin molecule.

Gelsolin derivatives are also contemplated for inclusion in embodiments of methods and compositions of the invention. A “functional derivative” of gelsolin is a derivative which possesses a bioactivity that is substantially similar to the bioactivity of gelsolin. By “substantially similar” is meant activity which may be quantitatively different but qualitatively the same. For example, a functional derivative of gelsolin could contain the same amino acid backbone as gelsolin but also contains other modifications such as post- translational modifications such as, for example, bound phospholipids, or covalently linked carbohydrate, depending on the necessity of such modifications for the performance of a therapeutic method of the invention. As used herein, the term is also meant to include a chemical derivative of gelsolin. Such derivatives may improve gelsolin's solubility, absorption, biological half-life, etc. The derivatives may also decrease the toxicity of gelsolin, or eliminate or attenuate any undesirable side effect of gelsolin, etc. Derivatives and specifically, chemical moieties capable of mediating such effects are disclosed in Remington, The Science and Practice of Pharmacy, 2012, Editor: Allen, Loyd V., Jr, 22^{nd} Edition). Procedures for coupling such moieties to a molecule such as gelsolin are well known in the art. The term “functional derivative” is intended to include the “fragments,” “variants,” “analogues,” or “chemical derivatives” of gelsolin.

### Microbial Infection

The terms “microbe” and “microbial” are used herein to reference a microorganism that causes a disease, which may be referred to herein as a “microbial infection”. The terms microbe and microbial encompass microorganisms such as, but not limited to: bacteria, fungi, viruses, and parasites that, when present in a subject, are capable of causing a bacterial, a fungal, a viral, and a parasitic infection, respectively. The term, “antimicrobial agent” as used herein in reference to treating or reducing an infection in a subject encompasses antibacterial agents, antifungal agents, antiviral agents, and anti-parasitic agents, which may be administered to a subject to treat a bacterial infection, a fungal infection, a viral infection, and a parasitic infection, respectively. The invention involves in some aspects, methods for treating infection in a subject. In some embodiments of the invention, a subject is known to have, is suspected of having been exposed, or is at risk of being exposed, or has been exposed to a microbial infection.

Characteristics of a microbial infection in a subject that may be assessed in control subjects or groups include but are not limited to: likelihood of survival, death, body weight, level of the microbe in a biological sample from the subject, presence of the microbe in a biological sample from the subject, presence, absence, and/or level of malaise, body temperature, fever, coughing, lung exudate, congestion, headache, chills, body aches, rash, flushing, etc. It will be understood that different characteristics may be indicated in different microbial infections and characteristics of a microbial infection in a human may differ from characteristics of the same microbial infection in another animal species. Characteristics present in different microbial infections and characteristics that present in humans and/or animals are known in the art. Those of skill in the art are able to readily select one or more characteristics of a microbial infection for detection and assessment in conjunction with use of methods and compositions of the invention. The term “characteristics” as used herein in reference to a microbial infection may refer to physiological symptoms of the microbial infection.

As used herein the terms “infection” and “microbial infection” refer to a disorder arising from the invasion of a host, superficially, locally, or systemically, by an infectious organism. Certain embodiments of methods and compositions of the invention may be used to treat microbial infections that arise in subjects due to infectious organisms such as microbes, including but not limited to bacteria, viruses, parasites, fungi, and protozoa.

### Microbial Agents

Microbial agents, which may also be referred to herein as pathogenic agents, may include bacterial agents, fungal agents, viral agents, parasitic agents, and protozoal agents. Microbial agents, such as those listed below herein, when present in a subject may result in a microbial infection in the subject.

Bacterial agents that can result in a bacterial infection when present in a subject may include gram-negative and gram-positive bacteria. Examples of gram-positive bacteria include Pasteurella species, Staphylococcus species including Staphylococcus aureus, Streptococcus species including Streptococcus pyogenes group A, Streptococcus viridans group, Streptococcus agalactiae group B, Streptococcus bovis, Streptococcus anaerobic species, Streptococcus pneumoniae, and Streptococcus faecalis, Bacillus species including Bacillus anthracis, Corynebacterium species including Corynebacterium diphtheriae, aerobic Corynebacterium species, and anaerobic Corynebacterium species, Diphtheroids species, Listeria species including Listeria monocytogenes, Erysipelothrix species including Erysipelothrix rhusiopathiae, Clostridium species including Clostridium perfringens, Clostridium tetani, and Clostridium difficile.

Gram-negative bacteria include Neisseria species including Neisseria gonorrhoeae and Neisseria meningitidis, Branhamella species including Branhamella catarrhalis, Escherichia species including Escherichia coli, Enterobacter species, Proteus species including Proteus mirabilis, Pseudomonas species including Pseudomonas aeruginosa, Pseudomonas mallei, and Pseudomonas pseudomallei, Klebsiella species including Klebsiella pneumoniae, Salmonella species, Shigella species, Serratia species, Acinetobacter species; Haemophilus species including Haemophilus influenzae and Haemophilus ducreyi, Brucella species, Yersinia species including Yersinia pestis and Yersinia enterocolitica, Francisella species including Francisella tularensis, Pasturella species including Pasteurella multocida, Vibrio cholerae, Flavobacterium species, meningosepticum, Campylobacter species including Campylobacter jejuni, Bacteroides species (oral, pharyngeal) including Bacteroides fragilis, Fusobacterium species including Fusobacterium nucleatum, Calymmatobacterium granulomatis, Streptobacillus species including Streptobacillus moniliformis, Legionella species including Legionella pneumophila.

Other types of bacteria include acid-fast bacilli, spirochetes, and actinomycetes.

Examples of acid-fast bacilli include Mycobacterium species including Mycobacterium tuberculosis and Mycobacterium leprae.

Examples of spirochetes include Treponema species including Treponema pallidum, Treponema pertenue, Borrelia species including Borrelia burgdorferi (Lyme disease), and Borrelia recurrentis, and Leptospira species.

Examples of actinomycetes include: Actinomyces species including Actinomyces israelii, and Nocardia species including Nocardia asteroides.

Viral agents that can result in a viral infection when present in a subject may include but are not limited to: Retroviruses, human immunodeficiency viruses including HIV-1, HDTV-III, LAVE, HTLV-III/LAV, HIV-III, HIV-LP, Cytomegaloviruses (CMV), Picornaviruses, polio viruses, hepatitis A virus, enteroviruses, human Coxsackie viruses, rhinoviruses, echoviruses, Calciviruses, Togaviruses, equine encephalitis viruses, rubella viruses, Flaviruses, dengue viruses, encephalitis viruses, yellow fever viruses, Coronaviruses, Rhabdoviruses, vesicular stomatitis viruses, rabies viruses, Filoviruses, ebola virus, Paramyxoviruses, parainfluenza viruses, mumps virus, measles virus, respiratory syncytial virus (RSV), Orthomyxoviruses, influenza viruses, Bungaviruses, Hantaan viruses, phleboviruses and Nairo viruses, Arena viruses, hemorrhagic fever viruses, reoviruses, orbiviruses, rotaviruses, Birnaviruses, Hepadnaviruses, Hepatitis B virus, parvoviruses, Papovaviridae, papilloma viruses, polyoma viruses, Adenoviruses, Herpesviruses including herpes simplex virus 1 and 2, varicella zoster virus, Poxviruses, variola viruses, vaccinia viruses, Irido viruses, African swine fever virus, delta hepatitis virus, non-A, non-B hepatitis virus, Hepatitis C, Norwalk viruses, astroviruses, and unclassified viruses.

Fungal agents that can result in a fungal infection when present in a subject may include, but are not limited to: Cryptococcus species including Crytococcus neoformans, Histoplasma species including Histoplasma capsulatum, Coccidioides species including Coccidiodes immitis, Paracoccidioides species including Paracoccidioides brasiliensis, Blastomyces species including Blastomyces dermatitidis, Chlamydia species including Chlamydia trachomatis, Candida species including Candida albicans, Sporothrix species including Sporothrix schenckii, Aspergillus species, and fungi of mucormycosis.

Parasitic agents that can result in a parasitic infection when present in a subject may include Plasmodium species, such as Plasmodium species including Plasmodium falciparum, Plasmodium malariae, Plasmodium ovale, and Plasmodium vivax and Toxoplasma gondii. Blood-borne and/or tissues parasites include Plasmodium species, Babesia species including babesia microti and Babesia divergens, Leishmania species including Leishmania tropica, Leishmania species, Leishmania braziliensis, Leishmania donovani, Trypanosoma species including Trypanosoma gambiense, Trypanosoma rhodesiense (African sleeping sickness), and Trypanosoma cruzi (Chagas' disease).

Other medically relevant microorganisms that may result in infections when present in a subject have been described extensively in the literature, e.g., see C. G. A Thomas, Medical Microbiology, Bailliere Tindall, Great Britain 1983, the entire contents of which is hereby incorporated by reference. Certain embodiments of methods and compositions of the invention may be used to treat infections by these and other medially relevant microorganisms.

### Antimicrobial Agents

Phrases such as “antimicrobial agent”, “antibacterial agent”, “antiviral agent,” “anti-fungal agent,” and “anti-parasitic agent,” have well-established meanings to those of ordinary skill in the art and are defined in standard medical texts. Briefly, anti-bacterial agents kill or inhibit the growth or function of bacteria. Anti-bacterial agents include antibiotics as well as other synthetic or natural compounds having similar functions. Antibiotics, typically, are low molecular weight molecules which are produced as secondary metabolites by cells, such as microorganisms. In general, antibiotics interfere with one or more bacterial functions or structures which are specific for the microorganism and which are not present in host cells.

A large class of anti-bacterial agents is antibiotics. Antibiotics that are effective for killing or inhibiting a wide range of bacteria are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Anti-bacterial agents are sometimes classified based on their primary mode of action. In general, anti-bacterial agents are cell wall synthesis inhibitors, cell membrane inhibitors, protein synthesis inhibitors, nucleic acid synthesis or functional inhibitors, and competitive inhibitors.

Anti-bacterial agents include but are not limited to aminoglycosides, β-lactam agents, cephalosporins, macrolides, penicillins, quinolones, sulfonamides, and tetracyclines. Examples of anti-bacterial agents include but are not limited to: Acedapsone, Acetosulfone Sodium, Alamecin, Alexidine, Amdinocillin Clavulanate Potassium, Amdinocillin, Amdinocillin Pivoxil, Amicycline, Amifloxacin, Amifloxacin Mesylate, Amikacin, Amikacin Sulfate, Aminosalicylic acid, Aminosalicylate sodium, Amoxicillin, Amphomycin, Ampicillin, Ampicillin Sodium, Apalcillin Sodium, Apramycin, Aspartocin, Astromicin Sulfate, Avilamycin, Avoparcin, Azithromycin, Azlocillin, Azlocillin Sodium, Bacampicillin Hydrochloride, Bacitracin, Bacitracin Methylene Disalicylate, Bacitracin Zinc, Bambermycins, Benzoylpas Calcium, Berythromycin, Betamicin Sulfate, Biapenem, Biniramycin, Biphenamine Hydrochloride, Bispyrithione Magsulfex, Butikacin, Butirosin Sulfate, Capreomycin Sulfate, Carbadox, Carbenicillin Disodium, Carbenicillin Indanyl Sodium, Carbenicillin Phenyl Sodium, Carbenicillin Potassium, Carumonam Sodium, Cefaclor, Cefadroxil, Cefamandole, Cefamandole Nafate, Cefamandole Sodium, Cefaparole, Cefatrizine, Cefazaflur Sodium, Cefazolin, Cefazolin Sodium, Cefbuperazone, Cefdinir, Cefditoren Pivoxil, Cefepime, Cefepime Hydrochloride, Cefetecol, Cefixime, Cefinenoxime Hydrochloride, Cefinetazole, Cefinetazole Sodium, Cefonicid Monosodium, Cefonicid Sodium, Cefoperazone Sodium, Ceforanide, Cefotaxime, Cefotaxime Sodium, Cefotetan, Cefotetan Disodium, Cefotiam Hydrochloride, Cefoxitin, Cefoxitin Sodium, Cefpimizole, Cefpimizole Sodium, Cefpiramide, Cefpiramide Sodium, Cefpirome Sulfate, Cefpodoxime Proxetil, Cefprozil, Cefroxadine, Cefsulodin Sodium, Ceftazidime, Ceftazidime Sodium, Ceftibuten, Ceftizoxime Sodium, Ceftriaxone Sodium, Cefuroxime, Cefuroxime Axetil, Cefuroxime Pivoxetil, Cefuroxime Sodium, Cephacetrile Sodium, Cephalexin, Cephalexin Hydrochloride, Cephaloglycin, Cephaloridine, Cephalothin Sodium, Cephapirin Sodium, Cephradine, Cetocycline Hydrochloride, Cetophenicol, Chloramphenicol, Chloramphenicol Palmitate, Chloramphenicol Pantothenate Complex, Chloramphenicol Sodium Succinate, Chlorhexidine Phosphanilate, Chloroxylenol, Chlortetracycline Bisulfate, Chlortetracycline Hydrochloride, Cilastatin, Cinoxacin, Ciprofloxacin, Ciprofloxacin Hydrochloride, Cirolemycin, Clarithromycin, Clavulanate Potassium, Clinafloxacin Hydrochloride, Clindamycin, Clindamycin Dextrose, Clindamycin Hydrochloride, Clindamycin Palmitate Hydrochloride, Clindamycin Phosphate, Clofazimine, Cloxacillin Benzathine, Cloxacillin Sodium, Cloxyquin, Colistimethate, Colistimethate Sodium, Colistin Sulfate, Coumermycin, Coumermycin Sodium, Cyclacillin, Cycloserine, Dalfopristin, Dapsone, Daptomycin, Demeclocycline, Demeclocycline Hydrochloride, Demecycline, Denofungin, Diaveridine, Dicloxacillin, Dicloxacillin Sodium, Dihydrostreptomycin Sulfate, Dipyrithione, Dirithromycin, Doxycycline, Doxycycline Calcium, Doxycycline Fosfatex, Doxycycline Hyclate, Doxycycline Monohydrate, Droxacin Sodium, Enoxacin, Epicillin, Epitetracycline Hydrochloride, Ertapenem, Erythromycin, Erythromycin Acistrate, Erythromycin Estolate, Erythromycin Ethylsuccinate, Erythromycin Gluceptate, Erythromycin Lactobionate, Erythromycin Propionate, Erythromycin Stearate, Ethambutol Hydrochloride, Ethionamide, Fleroxacin, Floxacillin, Fludalanine, Flumequine, Fosfomycin, Fosfomycin Tromethamine, Fumoxicillin, Furazolium Chloride, Furazolium Tartrate, Fusidate Sodium, Fusidic Acid, Gatifloxacin, Genifloxacin, Gentamicin Sulfate, Gloximonam, Gramicidin, Haloprogin, Hetacillin, Hetacillin Potassium, Hexedine, Ibafloxacin, Imipenem, Isoconazole, Isepamicin, Isoniazid, Josamycin, Kanamycin Sulfate, Kitasamycin, Levofloxacin, Levofuraltadone, Levopropylcillin Potassium, Lexithromycin, Lincomycin, Lincomycin Hydrochloride, Linezolid, Lomefloxacin, Lomefloxacin Hydrochloride, Lomefloxacin Mesylate, Loracarbef, Mafenide, Meclocycline, Meclocycline Sulfosalicylate, Megalomicin Potassium Phosphate, Mequidox, Meropenem, Methacycline, Methacycline Hydrochloride, Methenamine, Methenamine Hippurate, Methenamine Mandelate, Methicillin Sodium, Metioprim, Metronidazole Hydrochloride, Metronidazole Phosphate, Mezlocillin, Mezlocillin Sodium, Minocycline, Minocycline Hydrochloride, Mirincamycin Hydrochloride, Monensin, Monensin Sodium, Moxifloxacin Hydrochloride, Nafcillin Sodium, Nalidixate Sodium, Nalidixic Acid, Natamycin, Nebramycin, Neomycin Palmitate, Neomycin Sulfate, Neomycin Undecylenate, Netilmicin Sulfate, Neutramycin, Nifuradene, Nifuraldezone, Nifuratel, Nifuratrone, Nifurdazil, Nifurimide, Nifurpirinol, Nifurquinazol, Nifurthiazole, Nitrocycline, Nitrofurantoin, Nitromide, Norfloxacin, Novobiocin Sodium, Ofloxacin, Ormetoprim, Oxacillin Sodium, Oximonam, Oximonam Sodium, Oxolinic Acid, Oxytetracycline, Oxytetracycline Calcium, Oxytetracycline Hydrochloride, Paldimycin, Parachlorophenol, Paulomycin, Pefloxacin, Pefloxacin Mesylate, Penamecillin, Penicillin G Benzathine, Penicillin G Potassium, Penicillin G Procaine, Penicillin G Sodium, Penicillin V, Penicillin V Benzathine, Penicillin V Hydrabamine, Penicillin V Potassium, Pentizidone Sodium, Phenyl Aminosalicylate, Piperacillin, Piperacillin Sodium, Pirbenicillin Sodium, Piridicillin Sodium, Pirlimycin Hydrochloride, Pivampicillin Hydrochloride, Pivampicillin Pamoate, Pivampicillin Probenate, Polymyxin B Sulfate, Porfiromycin, Propikacin, Pyrazinamide, Pyrithione Zinc, Quindecamine Acetate, Quinupristin, Racephenicol, Ramoplanin, Ranimycin, Relomycin, Repromicin, Rifabutin, Rifametane, Rifamexil, Rifamide, Rifampin, Rifapentine, Rifaximin, Rolitetracycline, Rolitetracycline Nitrate, Rosaramicin, Rosaramicin Butyrate, Rosaramicin Propionate, Rosaramicin Sodium Phosphate, Rosaramicin Stearate, Rosoxacin, Roxarsone, Roxithromycin, Sancycline, Sanfetrinem Sodium, Sarmoxicillin, Sarpicillin, Scopafungin, Sisomicin, Sisomicin Sulfate, Sparfloxacin, Spectinomycin Hydrochloride, Spiramycin, Stallimycin Hydrochloride, Steffimycin, Sterile Ticarcillin

Disodium, Streptomycin Sulfate, Streptonicozid, Sulbactam Sodium, Sulfabenz, Sulfabenzamide, Sulfacetamide, Sulfacetamide Sodium, Sulfacytine, Sulfadiazine, Sulfadiazine Sodium, Sulfadoxine, Sulfalene, Sulfamerazine, Sulfameter, Sulfamethazine, Sulfamethizole, Sulfamethoxazole, Sulfamonomethoxine, Sulfamoxole, Sulfanilate Zinc, Sulfanitran, Sulfasalazine, Sulfasomizole, Sulfathiazole, Sulfazamet, Sulfisoxazole, Sulfisoxazole Acetyl, Sulfisoxazole Diolamine, Sulfomyxin, Sulopenem, Sultamicillin, Suncillin Sodium, Talampicillin Hydrochloride, Tazobactam, Teicoplanin, Temafloxacin Hydrochloride, Temocillin, Tetracycline, Tetracycline Hydrochloride, Tetracycline Phosphate Complex, Tetroxoprim, Thiamphenicol, Thiphencillin Potassium, Ticarcillin Cresyl Sodium, Ticarcillin Disodium, Ticarcillin Monosodium, Ticlatone, Tiodonium Chloride, Tobramycin, Tobramycin Sulfate, Tosufloxacin, Trimethoprim, Trimethoprim Sulfate, Trisulfapyrimidines, Troleandomycin, Trospectomycin Sulfate, Trovafloxacin, Tyrothricin, Vancomycin, Vancomycin Hydrochloride, Virginiamycin, Zorbamycin.

Anti-viral agents can be isolated from natural sources or synthesized and are useful for killing or inhibiting the growth or function of viruses. Anti-viral agents are compounds which prevent infection of cells by viruses or replication of the virus within the cell. There are several stages within the process of viral infection which can be blocked or inhibited by anti-viral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Anti-viral agents useful in the invention include but are not limited to: immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncytial virus), dideoxyinosine, dideoxycytidine, zidovudine (azidothymidine), imiquimod, and resimiquimod.

Anti-fungal agents are used to treat superficial fungal infections as well as opportunistic and primary systemic fungal infections. Anti-fungal agents are useful for the treatment and prevention of infective fungi. Anti-fungal agents are sometimes classified by their mechanism of action. Some anti-fungal agents function, for example, as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

Anti-parasitic agents kill or inhibit parasites. Examples of anti-parasitic agents, also referred to as parasiticides, useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, timidazole, trimethroprim-sulfamethoxazole, and tryparsamide some of which are used alone or in combination with others.

### Subjects

As used herein, a subject may be a vertebrate animal including but not limited to a human, mouse, rat, guinea pig, rabbit, cow, dog, cat, horse, goat, and primate, e.g., monkey. In certain aspects of the invention, a subject may be a domesticated animal, a wild animal, or an agricultural animal. Thus, the invention can be used to treat microbial infections in human and non-human subjects. For instance, methods and compositions of the invention can be used in veterinary applications as well as in human treatment regimens. In some embodiments of the invention, a subject is a human. In some embodiments of the invention, a subject has a microbial infection and is in need of treatment.

In some embodiments, a subject already has or had a microbial infection. In some embodiments, a subject is at an elevated risk of having an infection because the subject has one or more risk factors to have an infection. Risk factors for a microbial infection include: but are not limited to: immunosuppression, being immunocompromised, age, trauma, burns (e.g., thermal burns), surgery, foreign bodies, cancer, newborns, premature newborns, etc. A degree of risk of acquiring a microbial infection depends on the multitude and the severity or the magnitude of the risk factors that the subject has. Risk charts and prediction algorithms are available for assessing the risk of a microbial infection in a subject based on the presence and severity of risk factors. Other methods of assessing the risk of an infection in a subject are known by those of ordinary skill in the art.

As used herein in reference to when a subject infected with a microbial infection, the term “infected” means the day the subject is infected with the microbial infective agent, such as but not limited to: a bacterial agent, a viral agent, a fungal agent, a parasitic agent, etc. It will be understood that the day of a subject's known or potential exposure to a microbial agent may be regarded as day zero for the subject's infection with the microbial agent. Exposure to a microbial infection is understood to mean direct or indirect contact with an infected individual. A contact with an infected individual may be physical contact, contact with breath, saliva, fluid droplets, exudate, bodily fluid, discharge of an infected subject. In some embodiments, an indirect contact may be a physical contact by a subject with a substrate contaminated by the infected individual. Examples of substrates that may be contaminated by an infected individual include but are not limited to: food items, cloth, paper, metal, plastic, cardboard, fluids, air systems, etc. These and other means of exposure to microbial infections are known in the art.

### Assessments and Controls

A microbial infection in a subject can be detected using art-known methods, including but not limited to: assessing one or more characteristics of the microbial infection such as, but not limited to: presence of the microbe in a biological sample obtained from the subject; a level or amount of the microbe in a biological sample obtained from the subject; and presence and/or level of one or more physiological symptoms of the microbial infection detected in the subject. Characteristics of a microbial infection detected in a subject can be compared to control values of the characteristics of the microbial infection. A control value may be a predetermined value, which can take a variety of forms. It can be a single cut-off value, such as a median or mean. It can be established based upon comparative groups, such as in groups of individuals having the microbial infection and groups of individuals who have been administered a treatment for the microbial infection, etc. Another example of comparative groups may be groups of subjects having one or more symptoms of or a diagnosis of the microbial infection and groups of subjects without one or more symptoms of or a diagnosis of the microbial infection. The predetermined value, of course, will depend upon the particular population selected. For example, a population of individuals with the microbial infection that have been administered a gelsolin agent and not administered an antimicrobial agent, may have a one or more different characteristics of the microbial infection than a population of individuals having the microbial infection that have been administered the antimicrobial agent and not administered the gelsolin agent. Accordingly, the predetermined value selected may take into account the category in which an individual falls. Appropriate categories can be selected with no more than routine experimentation by those of ordinary skill in the art.

Controls can be used in methods of the invention to compare characteristics of different control groups, characteristics of a subject with those of a control group, etc. Comparisons between subjects and controls, one control with another control, etc. may be based on relative differences. For example, though not intended to be limiting, a physiological symptom in a subject treated with a synergistic therapeutic method of the invention comprising administering to the subject a gelsolin agent and an antimicrobial agent, can be compared to the physiological symptom of a control group that has been administered the gelsolin agent and not administered the antimicrobial agent. The comparison may be expressed in relative terms, for example, if elevated body temperature (indicative of fever), or a reduced body temperature, is a characteristic of a microbial infection, a body temperature of a subject treated with a synergistic therapeutic method of the invention may be compared to a control level of body temperature. In some embodiments, a suitable control is a subject not treated with a synergistic therapeutic method of the invention. A comparison of a treated versus a control may include comparing percentage temperature differences between the treated subject and the selected control. In some instances, a body temperature of a subject treated with a method of the invention may be determined to be low relative to a selected control, with the comparison indicating a 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, or 5.9% lower body temperature in the subject as compared to the body temperature level in the control.

In some certain instances, a body temperature of a subject treated with a method of the invention may be determined to be higher relative to a selected control, with the comparison indicating a 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, or 5.9% higher lower body temperature in the subject as compared to the body temperature level in the control.

In another non-limiting example, a level of a microbial infection can be determined using an assay to detect the presence, absence, and/or amount of the microbe in a biological sample that is obtained from a subject having the microbial infection. The results of the assay in a subject treated using a synergistic therapeutic method of the invention can be compared to a control level of the microbial infection, for example results of the assays on a sample obtained from a control subject not having been so treated. Results of assays to assess a level of a microbial infection in a subject treated using a method of the invention can be compared to a control to determine a percentage difference between the subject and the control levels. In some embodiments, a level of a treated subject's microbial infection is less than 100% of a control infection level. In certain embodiments of the invention the level of the treated subject's microbial infection is less than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%. 0.9%, 0.8%, 0.7%, 0.6%, 0.5%, 0.4%, 0.3%, 0.2%, or 0.1% of the control level of the microbial infection.

In another non-limiting example, a level of a microbial infection and/or increase in a therapeutic effect of an antimicrobial agent using a method of the invention can be determined by comparing a likelihood of survival of a subject treated with a synergistic method or composition of the invention with a control likelihood of survival. A non-limiting example of a control likelihood of survival is the likelihood of survival in a subject with a microbial infection not treated with a method of the invention. Non-limiting examples of parameters of likelihood of survival that can be measured include: determination of length of time (hours, days, weeks, etc.) a subject remains alive following a treatment of the invention, and whether a subject dies or survives following a treatment of the invention. It will be understood how these and other parameters relating to likelihood of survival can be compared to controls to assess and determine therapeutic effectiveness of a synergistic method or composition of the invention. A non-limiting example of a control of likelihood of survival is the number of days a subject survives after treatment with a synergistic method of the invention compared to the control number of days of survival in the absence of the administration of the synergistically effective amount of each of the antimicrobial agent and the gelsolin agent. In some embodiments of the invention a likelihood of survival of a subject treated with a synergistic method of the invention is at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 125%, 150%, 175%, 200%, 300%, 400%, 500%, higher than a control likelihood of survival.

In another non-limiting example, a level of a microbial infection and/or increase in a therapeutic effect of an antimicrobial agent using a method of the invention can be determined by comparing level of lung pathology in a subject treated with a synergistic method or composition of the invention with a control level of lung pathology. A non- limiting example of a control level of lung pathology is the level of lung pathology in a subject with a microbial infection not treated with a method of the invention. Non-limiting examples of parameters of lung pathology that can be measured include: determination of lung histopathology in a subject. In a non-limiting examples, histopathology of lung tissue (for example obtained via biopsy from a subject, etc.) can be assessed using art-known methods, for example, the lung tissue may be observed and scored in a blinded fashion by a board-certified pathologist. A scoring system can be used to compare a subject's lung tissue with a control. In a non-limiting example, a four-point, four-criteria system (inflammation; infiltrate; necrosis; and other, including hemorrhage) with a maximum score of 16 points may be used to evaluate lung pathology. Points for each criterion can be assigned based as no (0), minimal (1), mild (2), moderate (3), and severe (4) pathologic findings. The scoring system permits comparison of subject tissue with control tissue to assess lung pathology. Additional means of comparing lung pathology are known in the art and may be used in conjunction with methods of the invention. In some embodiments of the invention a level of lung pathology of a subject treated with a synergistic method of the invention is at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 125%, 150%, 175%, 200%, 300%, 400%, 500%, lower than a control level of lung pathology.

In another non-limiting example, microbial infection and/or increase in a therapeutic effect of an antimicrobial agent using a method of the invention can be determined by comparing level of a level of a weight loss or relative weight loss in a subject treated with a synergistic method or composition of the invention with a control level of weight loss or relative weight loss. A non-limiting example of a control level of weight loss is a level of weight loss in a subject with a microbial infection not treated with a method of the invention. Non-limiting examples of parameters of weight loss and/or relative weight loss that can be measured include: a subject's weight prior to a microbial infection, a subject's weight during a microbial infection prior to treatment with a synergistic method of the invention, a subject's weight after receiving a synergistic therapeutic method of the invention, etc. In a non-limiting examples, a weight of a subject with a Pseudomonas aeruginosa infection can be determined before and after administration of a synergistic treatment of the invention comprising a gelsolin agent and a carbapenem class agent, a non-limiting example of which is meropenem. The subject's weight can be compared to the subject's pretreatment weight, pre-infection weight, and/or another control weight. A reduction in weight loss in the subject following the administration of the synergistic treatment of the invention, indicates a reduction in the microbial infection in the subject. In some embodiments of the invention a level of weight loss in a subject treated with a synergistic method of the invention is at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 125%, 150%, 175%, 200%, 300%, 400%, 500%, lower than a control level of weight loss.

It will be understood that controls may be, in addition to predetermined values, samples of materials tested in parallel with the experimental materials. Examples include samples from control populations or control samples generated through manufacture to be tested in parallel with the experimental samples; and also a control may be a sample from a subject prior to, during, or after a treatment with an embodiment of a method or composition of the invention. Thus one or more characteristics determined for a subject having an infection may be used as “control” values for those characteristics in that subject at a later time.

In some embodiments of the invention effectiveness of a synergistic method of the invention can be assessed by comparing synergistic therapeutic results in a subject treated using a method of the invention to one or both of: (1) an individual therapeutic effect of the gelsolin agent and (2) an individual therapeutic effect of the antimicrobial agent. In certain aspects of the invention, a difference in a level of therapeutic effectiveness may be assessed on a scale indicating an increase from a control level. In some aspects an increase is from a control level of zero obtained in (1) or (2) to a level greater than zero resulting from treatment with a synergistic method of the invention. In some embodiments of the invention, a level of therapeutic effect of a synergistic therapeutic method of the invention is an increase by at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, 100%, 125%, 150%, 175%, 200%, 300%, 400%, 500%, or more from a control level of therapeutic effect.

### Delayed Dosing Methods

Some embodiments of the invention include a delayed dosing schedule that has been determined to be effective in reducing viral infection in an infected subject. A delay in administering a gelsolin agent to a subject until three or more days following the day (day zero) the subject is infected with a viral infection enhances the therapeutic effect of the gelsolin agent. Some embodiments of treatment methods of the invention include administering to a subject having a viral infection an effective amount of a gelsolin agent, wherein the gelsolin agent is administered at least 3, 4, 5, 6, 7, 8, 9, or more days after infection of the subject with the viral infection. In some embodiments the gelsolin agent is not administered to the subject the day the subject is infected with the viral infection (day zero). In in some embodiments the gelsolin agent is not administered on the first day (day 1) after the day a subject is infected with the viral infection. In some embodiments the gelsolin agent is not administered on the second day (day two) after the subject is infected with the viral infection. In some embodiments of methods of the invention, a gelsolin agent is not administered on one or more of day zero, day one, and day two of a viral infection in a subject.

As used herein in reference to when a subject infected with a microbial infection, the term “infected” means the day the subject is infected with the microbial infective agent, for example but not limited to the bacterial agent, the viral agent, the fungal agent, etc. It will be understood that the day of a subject's known or possible exposure to a microbial agent may be regarded as day zero for the subject's infection with the microbial agent.

Art-known standard regimens to treat viral infections may include one or more of: (1) administering an antiviral to a subject on the day of a known or potential exposure of the subject to the virus, (2) administering an antiviral to a subject within 48 hours of a known or potential exposure of the subject to the virus, (3) seasonal prophylaxis with the antiviral by administering the antiviral to the subject without a specific known exposure to the virus, and (4) prophylaxis with an antiviral in situations of community outbreak of a virus. Exposure to a viral infection will be understood to mean direct or indirect contact with an individual infected with the viral infection. Non-limiting examples of contact with an infected individual may be physical contact, contact with breath, saliva, fluid droplets, exudate, bodily fluid, discharge of an infected subject, etc. In some embodiments, an indirect contact may be a physical contact by a subject with a substrate contaminated by the infected individual. Examples of substrates that may be contaminated by an individual infected with a viral infection include but are not limited to: food items, cloth, paper, metal, plastic, cardboard, fluids, air systems, etc. These and other means of exposure to viral infections are known in the art. Methods of the invention may be used to treat viral infections such as: Influenza A, B, C, and D infections. Non-limiting examples of viral infections include those caused by H1N1, H3N2, Coronaviruses (for example: 229E, NL63, OC43, HKUI, MERS-COV, SARS-CoV, SARS-COV-2, etc.)

Methods of treating a viral infection using a timed/delayed gelsolin agent dosing regimen may include administration of a gelsolin agent at a determined time delay following a subject's known exposure to a viral infection, suspected exposure to a viral infection, potential exposure to a viral infection, and/or risk of exposure to a viral infection. A gelsolin agent administered may include a gelsolin molecule, a functional fragment thereof, or a functional derivative of the gelsolin molecule. In some embodiments a gelsolin molecule is a plasma gelsolin (pGSN), and in certain embodiments of methods of the invention, a gelsolin molecule is a recombinant gelsolin molecule.

In some embodiments, an effective amount of a gelsolin agent has an increased therapeutic effect against the viral infection in the subject, compared to a control therapeutic effect, wherein the control therapeutic effect includes a therapeutic effect that results when the gelsolin agent is not administered to the subject. In some embodiments, a therapeutic effect of an administered gelsolin agent is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% greater than a control therapeutic effect.

In certain methods of the invention, a therapeutic effect of the administration of the gelsolin agent reduces a level of a viral infection in a subject compared to a control level of the viral infection, wherein the control level of infection may be a level of infection in the absence of administering the gelsolin agent. In some embodiments of the invention, a level of a subject's viral infection following administration of a gelsolin agent in a method of the invention is at least 5%, 10%, 15%, 20%, 25%, 30%, 35%, 40%, 45%, 50%, 55%, 60%, 65%, 70%, 75%, 80%, 85%, 90%, 95%, or 100% lower than a control level of viral infection.

One or more levels of a viral infection in a subject may be determined using one or more of: an assay to detect for example, presence, absence, and/or level of a characteristic of the viral infection in a biological sample obtained from the subject; observing the subject; assessing one or more physiological symptoms of the viral infection in the subject; assessing a likelihood of survival of the subject; or other art-known means. Physiological symptoms of a viral infection may include, but are not limited to: one or more of: fever, malaise, weight loss, and death.

An embodiment of the invention may include administering an effective amount of a gelsolin agent to a subject at day 3, 4, 5, 6, 7, or more following the subject's exposure or suspected exposure to a viral infection in which the administration of the effective amount of the gelsolin agent increases the subject's likelihood of survival compared to a control likelihood of survival, wherein the control likelihood of survival is a likelihood of survival in the absence of the administration of the gelsolin agent. An increase in a subject's likelihood of survival following administration of a gelsolin agent using a timed dosing regimen of the invention is at least 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, or 200% higher than the control likelihood of survival.

A time-delay in administering a gelsolin agent to a subject until three or more days following the day (day zero) the subject is infected with a viral infection enhances the therapeutic effect of the gelsolin agent and can be used in conjunction with administration of an antiviral agent, resulting in a synergistic effect of the antiviral agent and the gelsolin agent administered to a subject. In some aspects of the invention, a method of treating a viral infection of the invention includes administering to a subject an antiviral agent one or more days prior to a time-delayed administering of a gelsolin agent to the subject. In some embodiments an antiviral agent may be administered prior to a subject's exposure or potential exposure to a viral infection, or may be administered on day zero, day one, day two of exposure to or suspected exposure of the subject to the viral infection. It has been identified that an effective amount of each of a gelsolin agent and an antiviral agent administered to the subject may have a synergistic therapeutic effect against the viral infection, compared to a control therapeutic effect, in which a gelsolin agent and an antiviral agent are not both administered to the subject in a manner resulting in a synergistic effect. It will be understood that as described elsewhere herein, an antiviral agent is administered in a clinically acceptable amount and a control therapeutic effect may be a therapeutic effect of administering a clinically acceptable amount of the antiviral agent administered without administering the gelsolin agent.

In some embodiments of methods of the invention, a clinically acceptable amount of the antiviral agent is an amount below a maximum tolerated dose (MTD) of the antiviral agent. In some instances, an MTD of the antiviral agent is a highest possible but still tolerable dose level of the antiviral agent for the subject. In certain instances, an MTD of the antiviral agent is determined at least in part on a pre-selected clinical-limiting toxicity for the antiviral agent. In methods of the invention that include administering synergistically effective amount of a gelsolin agent and an antiviral agent, the synergistic effect decreases a minimum effective dose (MED) of the antiviral agent in the subject. In certain methods of the invention, an MED is a lowest dose level of the antiviral agent that provides a clinically significant response in average efficacy, wherein the response is statistically significantly greater than a response provided by a control that does not include the dose of the antiviral agent.

Non-limiting examples of antiviral agents that may be administered to a subject as part of an antiviral regimen are: neuraminidase inhibitor antiviral drugs: oseltamivir phosphate, (available as a generic version or under the trade name Tamiflu), zanamivir (trade name Relenza®), and peramivir (trade name Rapivab®); and cap-dependent endonuclease (CEN) inhibitors such as: baloxavir marboxil (trade name Xofluza®).

Antiviral therapies for preventing and treating viral infections such as Influenza A, B, C, and D infections are known and routinely used in the art. It is also recognized that certain viral strains may be resistant to known antiviral therapies [see for example Moscona, A., 20090, N Engl J Med 360; 10:953-956]. Some embodiments of methods of the invention increase efficacy of an antiviral agent to treat a viral infection caused by a viral strain that is not resistant to an anti-viral agent. Certain embodiments of methods of the invention increase efficacy of an antiviral agent to treat a viral infection caused by a viral strain that is resistant to the antiviral agent.

Certain embodiments of methods of the invention treat a viral infection using a timed dose gelsolin regimen administered in the absence of a regimen of administering an antiviral agent. Some embodiments of methods of the invention treat a viral infection by administering an antiviral agent regimen and a time-delayed gelsolin regimen to a subject in need of such treatment. In some embodiments of methods of the invention administration to a subject of an antiviral agent and a delayed-dose gelsolin agent result in synergistic therapeutic effect of the gelsolin agent and the antiviral agent in the subject. A synergistic therapeutic effect of certain embodiments of methods of the invention can enhance treatment of a non-antiviral-resistant viral infection in a subject as compared to a control therapeutic effect. A synergistic therapeutic effect of some embodiments of methods of the invention can be used to enhance treatment of an antiviral-resistant viral infection in a subject as compared to a control therapeutic effect.

### Preparation and Administration of Pharmacological Agents

Methods and compositions of the invention have important implications for patient treatment and also for the clinical development of new therapies. It is also expected that clinical investigators now will use the present methods for determining entry criteria for human subjects in clinical trials. Health care practitioners select therapeutic regimens for treatment based upon the expected net benefit to the subject. The net benefit is derived from the risk to benefit ratio.

The amount of a treatment may be varied for example by increasing or decreasing the amount of gelsolin agent and/or antimicrobial agent administered to a subject, by changing the therapeutic composition administered, by changing the route of administration, by changing the dosage timing and so on. The effective amount will vary with the particular infection or condition being treated, the age and physical condition of the subject being treated, the severity of the infection or condition, the duration of the treatment, the specific route of administration, and like factors are within the knowledge and expertise of the health practitioner. For example, an effective amount can depend upon the degree to which an individual has been exposed to or affected by exposure to the microbial infection.

### Effective Amounts

The term “effective amount” as used herein in relation to a treatment method or composition of the invention, is referred to as a “synergistically effect amount”. Methods of the invention comprise administering each of a gelsolin agent and an antimicrobial agent in amounts that are synergistically effective amounts of the gelsolin agent and the antimicrobial agent. When administered to a subject in a method of the invention, synergistically effective amounts of the gelsolin agent and the antimicrobial agent result in a synergistic therapeutic effect against and/or a reduction in the microbial infection in the subject.

An effective amount is a dosage of each of the pharmacological agents sufficient to provide a medically desirable result. Examples of pharmacological agents that may be used in certain embodiments of compositions and methods of the invention include, but are not limited to: gelsolin agents and antimicrobial agents. It should be understood that pharmacological agents of the invention are used to treat or prevent infections, that is, they may be used prophylactically in subjects at risk of developing an infection. Thus, an effective amount is that amount which can lower the risk of, slow or perhaps prevent altogether the development of an infection. It will be recognized when the pharmacologic agent is used in acute circumstances, it is used to prevent one or more medically undesirable results that typically flow from such adverse events.

Factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention is that amount effective to treat the disorder, such as an infection. In the case of infections the desired response is inhibiting the progression of the infection and/or reducing the level of the infection. This may involve only slowing the progression of the infection temporarily, although it may include halting the progression of the infection permanently. This can be monitored by routine diagnostic methods known to those of ordinary skill in the art. The desired response to treatment of the infection also can be delaying the onset or even preventing the onset of the infection.

### Pharmaceutical Agents and Delivery

The pharmacological agents used in the methods of the invention are preferably sterile and contain an effective amount of gelsolin and an effective amount of an antimicrobial agent for producing the desired response in a unit of weight or volume suitable for administration to a subject. Doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual physician or veterinarian, particularly in the event of any complication. A therapeutically effective amount typically varies from 0.01 mg/kg to about 1000 mg/kg, from about 0.1 mg/kg to about 200 mg/kg, or from about 0.2 mg/kg to about 20 mg/kg, in one or more dose administrations daily, for one or more days. Gelsolin agents and an antimicrobial agents may also be referred to herein as pharmacological agents.

Various modes of administration are known to those of ordinary skill in the art which effectively deliver the pharmacological agents of the invention to a desired tissue, cell, or bodily fluid. The manner and dosage administered may be adjusted by the individual physician, healthcare practitioner, or veterinarian, particularly in the event of any complication. The absolute amount administered will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. In certain embodiments of the invention, such preparations may contain salt, buffering agents, preservatives, compatible carriers, aqueous solutions, water, etc. When used in medicine, the salts may be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Various modes of administration known to the skilled artisan can be used to effectively deliver pharmaceutical composition of the invention that comprises an antimicrobial agent and a gelsolin agent to a subject to produce a synergistic therapeutic effect against a microbial infection in the subject. Methods for administering such a composition or pharmaceutical compound of the invention may be topical, intravenous, oral, intracavity, intrathecal, intrasynovial, buccal, sublingual, intranasal, transdermal, intravitreal, subcutaneous, intramuscular and intradermal administration. In some embodiments of the invention a means for administering a composition of the invention is inhalation. The invention is not limited by the particular modes of administration disclosed herein. Standard references in the art (e.g., Remington, The Science and Practice of Pharmacy, 2012, Editor: Allen, Loyd V., Jr, 22^{nd} Edition) provide modes of administration and formulations for delivery of various pharmaceutical preparations and formulations in pharmaceutical carriers. Other protocols which are useful for the administration of a therapeutic compound of the invention will be known to a skilled artisan, in which the dose amount, schedule of administration, sites of administration, mode of administration (e.g., intra-organ) and the like vary from those presented herein. Other protocols which are useful for the administration of pharmacological agents of the invention will be known to one of ordinary skill in the art, in which the dose amount, schedule of administration, sites of administration, mode of administration and the like vary from those presented herein.

Administration of pharmacological agents of the invention to mammals other than humans, e.g. for testing purposes or veterinary therapeutic purposes, is carried out under substantially the same conditions as described above. It will be understood by one of ordinary skill in the art that this invention is applicable to both human and animal diseases. Thus, this invention is intended to be used in husbandry and veterinary medicine as well as in human therapeutics. A pharmacological agent may be administered to a subject in a pharmaceutical preparation.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, pills, lozenges, each containing a predetermined amount of the active compound (e.g., gelsolin). Other compositions include suspensions in aqueous liquids or non- aqueous liquids such as a syrup, elixir, an emulsion, or a gel.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N. Y., pp. 367-383; Newmark, et al., 1982, J. Appl. Biochem. 4:185-189. Other polymers that could be used are poly-1,3-dioxolane and poly-1,3,6-tioxocane.

For the pharmacological agent the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of gelsolin agent and/or the antimicrobial agent or by release of the biologically active material beyond the stomach environment, such as in the intestine.

Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of gelsolin. Gelsolin is delivered to the lungs of a mammal while inhaling and traverses across the lung epithelial lining to the blood stream.

Nasal (or intranasal) delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran. The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

Pharmacological agent(s), including specifically but not limited to a gelsolin agent and an antimicrobial agent, may be provided in particles. Particles as used herein means nano or microparticles (or in some instances larger) which can consist in whole or in part of gelsolin or the antimicrobial agent as described herein. The particles may contain the pharmacological agent(s) in a core surrounded by a coating, including, but not limited to, an enteric coating. The pharmacological agent(s) also may be dispersed throughout the particles. The pharmacological agent(s) also may be adsorbed into the particles. The particles may be of any order release kinetics, including zero order release, first order release, second order release, delayed release, sustained release, immediate release, and any combination thereof, etc. The particle may include, in addition to the pharmacological agent(s), any of those materials routinely used in the art of pharmacy and medicine, including, but not limited to, erodible, nonerodible, biodegradable, or nonbiodegradable material or combinations thereof. The particles may be microcapsules which contain the gelsolin in a solution or in a semi-solid state. The particles may be of virtually any shape.

Both non-biodegradable and biodegradable polymeric materials can be used in the manufacture of particles for delivering the pharmacological agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The pharmacological agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the pharmacological agent(s) for at least 7 days, and preferably 30-60 days. Long-term sustained release implants are well-known to those of ordinary skill in the art and include some of the release systems described above.

The invention also contemplates the use of kits. In some aspects of the invention, the kit can include one or more pharmaceutical preparation vial, a pharmaceutical preparation diluent vial, an antimicrobial agent and a gelsolin agent. A vial containing the diluent for the pharmaceutical preparation is optional. A diluent vial may contain a diluent such as physiological saline for diluting what could be a concentrated solution or lyophilized powder of the gelsolin agent and/or the antimicrobial agent. The instructions can include instructions for mixing a particular amount of the diluent with a particular amount of the concentrated pharmaceutical preparation, whereby a final formulation for injection or infusion is prepared. The instructions may include instructions for treating a subject with effective amounts of the gelsolin agent and the antimicrobial agent. It also will be understood that the containers containing the preparations, whether the container is a bottle, a vial with a septum, an ampoule with a septum, an infusion bag, and the like, can contain indicia such as conventional markings that change color when the preparation has been autoclaved or otherwise sterilized.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

Antibiotic resistant pneumococcal pneumonia occur and can be problematic. Studies have been conducted to assess a novel therapeutic strategy for combating infections that includes means to augment innate immunity. Experiments were performed to determine the effect of pGSN administration on macrophages and host survival.

### Methods

### Bacterial Strains and Culture

S. pneumoniae serotype 3 (catalog no. 6303, American Type Culture Collection, Rockville, MD) were cultured overnight on 5% sheep blood-supplemented agar petri dishes (catalog no. 90001-282, VWR, West Chester, PA) and prepared and quantified as previously reported (Yang Z. et al., Am J Physiol Lung Cell Mol Physiol 2015; 309: L11-6).

In vitro and In vivo Procedures

### (1) In Vitro Studies

In vitro studies were performed in which 125-250 μg/ml pGSN was added to bacterial cultures and bacterial survival was determined.

### (2) In Vivo Studies

B16 mice were challenged with 10⁵ pneumococci by i.n. insufflation and were administered 10 mg pGSN s.c. 2 h before and 8 and 20 h after the infection. In some studies the pGSN was administered as an aerosol for 15 or 30 minutes prior to infection. The aerosol was generated as in Hamada, K., et al, J. Immunology. 2003; 170 (4): 1683-9, using a solution of 5 mg/ml.

### Results/Discussion

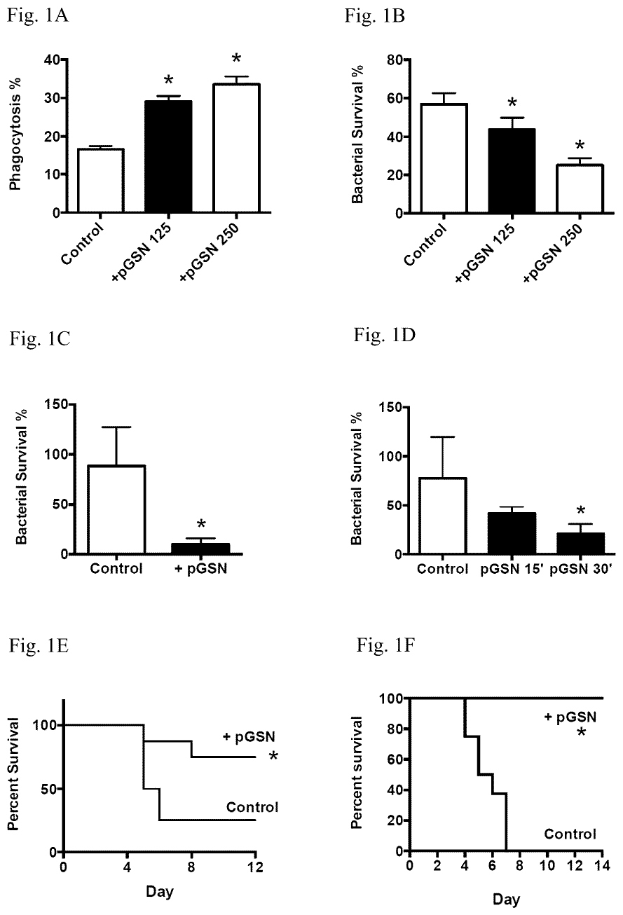

Results of in vitro studies demonstrated that pGSN improved macrophage uptake (FIG. 1A) and killing of internalized pneumococci (FIG. 1B) when present at 125-250 μg/ml, which is similar to normal plasma levels. In vivo, pGSN (10 mg s.c. 2 h before and 8 and 20 h after infection improved bacterial clearance (fewer surviving bacteria at 24 h) in B16 mice challenged with 10⁵ pneumococci by i.n. insufflation (FIG. 1C); similar results were seen when pGSN was administered as an aerosol for 15 or 30 minutes prior to infection; aerosol generated as in Hamada, K., et al, J. Immunology. 2003; 170(4): 1683-9 using a solution of 5 mg/ml (FIG. 1D). Systemic pGSN (s.c.) improves survival in primary (FIG. 1E, using 3×10⁵ CFU inoculum) or secondary post-influenza pneumococcal pneumonia (FIG. 1F, using 500 CFU inoculum on day 7 after mild influenza infection with PR8) even in the absence of any antibiotic treatment. *=p<0.05 vs control, n=6-12 per group. All experiments used serotype 3 Strep. Pneumoniae [ATCC #6303].

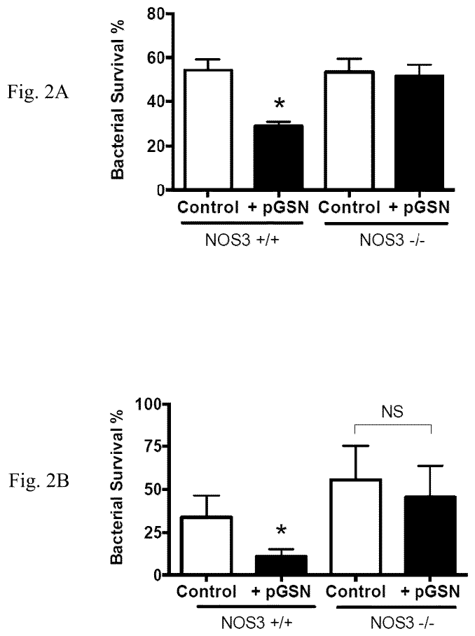

Macrophage NOS3 is an important mechanism for host defense against pneumonia in mice, and also functions in human macrophages (Yang, Z., et al., Elife. 2014;3. Epub 2014 Oct. 16. Doi 10.7554/elife.03711). Results indicated that this pathway functions as an important mechanism for pGSN effects on macrophages, because the pGSN was unable to improve bacterial killing response in NOS3-deficient macrophages (FIG. 2A) and in NOS3 deficient mice (FIG. 2B).

Additional studies were performed using E. Coli and Francisella tularensis (see Yang. Z., et al, American Journal of Physiology Lung Cellular and Molecular Physiology. 2015;309(1):L11-6).

### Example 2

Studies were performed to evaluate effects of pGSN treatment on antibiotic-sensitive and antibiotic-resistant mouse models of pneumococcal pneumonia.

### Methods

### Bacterial Strains and Culture

S. pneumoniae serotypes 3 and 14 (Catalog nos. 6303 and 700677, respectively) were obtained from the American Type Culture Collection (Rockville, MD). Serotype 3 bacteria were cultured overnight on 5% sheep blood-supplemented agar petri dishes (Catalog no. 90001-282, VWR, West Chester, PA) and prepared and quantified as previously reported (Yang Z. et al., Am J Physiol Lung Cell Mol Physiol 2015; 309:L11-6). Because serotype 14 required a more detailed protocol to achieve consistent results, the growth protocol reported in Restrepo AV et al., BMC Microbiol 2005; 5:34 was followed, which uses two sequential expansions in liquid broth culture before centrifugation and adjustment of bacterial concentration by OD600 for in vivo administration.

### Mouse Models of Pneumococcal Pneumonia

Normal 6-to 8-week (wk) old male CD1 mice were obtained from Charles River Laboratories (Wilmington, MA). Primary pneumococcal pneumonia was induced as previously reported (Yang Z. et al., Am J Physiol Lung Cell Mol Physiol 2015; 309:L11-6.) For antibiotic-sensitive pneumonia, intranasal instillation of 1.5-2×10⁶ colony-forming units (CFU) of Streptococcus pneumoniae type 3 was performed into mice under anesthesia with ketamine (72 mg/kg i.p.) plus xylazine (9.6 mg/kg i.p.). Streptococcus pneumoniae type 14, which is resistant to penicillin (minimum inhibitory concentration (MIC)=8 μg/ml) and other antibiotics (Jabes D. et al., J Infect Dis 1989; 159:16-25), was used to model antibiotic-resistant pneumonia. For this pathogen, range-finding experiments identified a high lethality inoculum of approximately 300×10⁶ colony-forming units (CFU) which was used for instillation under anesthesia as above. Most trials used 10 mice per group for the vehicle, penicillin (PEN), pGSN or PEN+pGSN groups.

### Treatments and Outcomes

Recombinant human pGSN (rhu-pGSN) was synthesized in E. coli and purified by Fujifilm Diosynth (Billingham, UK). rhu-pGSN was administered to mice by intraperitoneal injection at doses ranging from 5-10 mg as detailed in Results. In some experiments, penicillin (G Procaine Injectable Suspension, NDC 57319-485-05, Phoenix Pharmaceuticals) was administered by i.m. injection of 0.1-2 mg. The mice were monitored for 10 days, measuring survival, changes in weight and overall morbidity using a composite index (i.e., 1 point each for hunched appearance, ruffled fur or partly closed eyes; 1.5 points for prolapsed penis or splayed hind quarter; 2 points for listlessness, with a maximum score of 8; the assessment was performed without blinding to treatment group) adapted from guidelines in Burkholder T. et al., Curr Protoc Mouse Biol 2012; 2:145-65. Weights and morbidity scores for the last day alive were carried forward for animals that did not survive. To assess lung inflammation by quantifying neutrophil influx, one cohort of animals underwent lung lavage at 48 hours following infection after euthanasia as previously described (Yang Z. et al., Am J Physiol Lung Cell Mol Physiol 2015; 309: L11-6; and Yang Z. et al., Elife 2014; 3. After centrifugation, resuspended lavage samples were counted by hemocytometer and differential cell counts were performed on Wright-Giemsa stained cytocentrifuge preparations.

### Statistical Analysis

Data were analyzed using Prism (GraphPad Software) or SAS (SAS Institute) software. Differences in Kaplan-Meier survival curves were analyzed using a log-rank test with Sidak adjustment for multiple comparisons. For other measurements, differences between groups were examined by ANOVA.

### Results

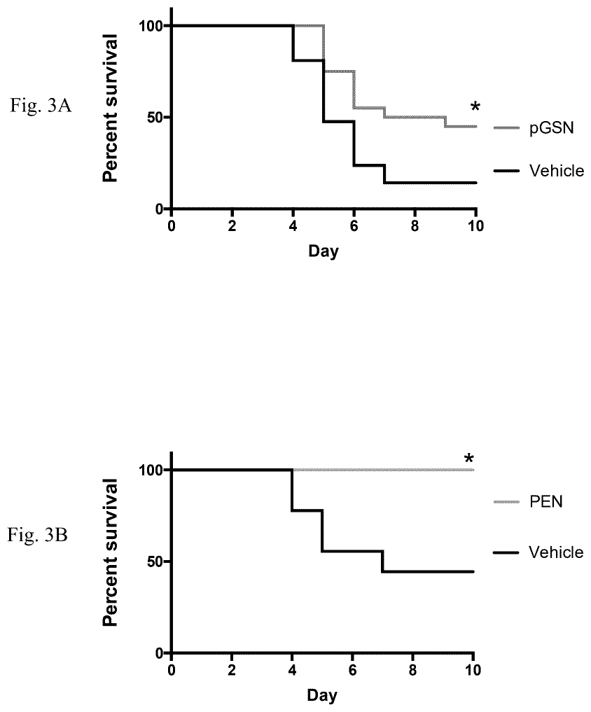

Delayed treatment with rhu-pGSN was tested in the same murine model used previously to demonstrate improved survival with pre-treatment (Yang Z. et al., Am J Physiol Lung Cell Mol Physiol 2015; 309:L11-6). As shown in FIG. 3A, pGSN treatment given only on days 2 and 3 after infection with serotype 3 pneumococci led to substantially improved survival from a highly lethal inoculum compared to vehicle controls, even in the absence of antibiotic treatment. To contrast with subsequent experiments using serotype 14, the 100% survival of antibiotic-treated mice confirmed that serotype 3 was highly sensitive to penicillin (FIG. 3B).

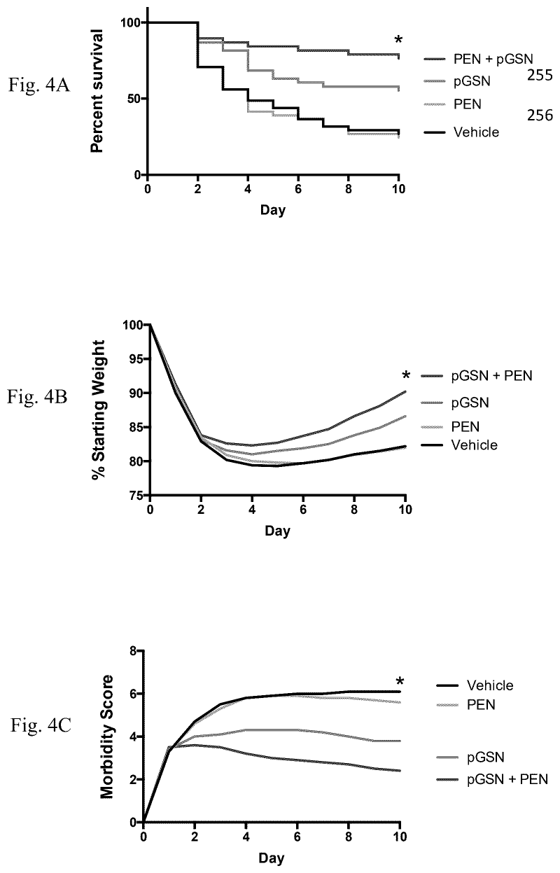

To determine if these findings extended to an antibiotic-resistant pneumonia, a similar model was developed using highly virulent serotype 14 pneumococci. Treatments were begun at 24 hours after infection and continued daily for 9 days. Mice treated with only the diluent vehicle experienced high mortality (FIG. 4A). Penicillin treatment alone had no benefit (FIG. 4A), consistent with the reported in vitro high-level resistance of this bacterial strain (Jabes D. et al., J Infect Dis 1989; 159:16-25).

During the 24 hours prior to treatment, all the mice experienced identical deterioration evidenced by equivalent weight loss and morbidity scores. Neutrophil influx at 48 hours after infection was decreased in animals treated with a single dose of pGSN with or without penicillin (total lavage neutrophils×10E4 in vehicle, PEN, pGSN and PEN+pGSN groups respectively: 186±54, 153±74, 111±16, 104±20; p<03, n=5-6/group). rhu-pGSN treatment alone caused substantial improvement in overall survival, recovery from weight loss, and improvement in morbidity scoring (FIGS. 4A-C).

In vitro, penicillin treatment alone or in combination with pGSN had no effect on bacterial growth (increase in bacterial CFU, 1 hour (h) culture with vehicle, PEN (16 μg/ml) or PEN+pGSN (250 μg/ml): 88000, 105000, 88000, respectively, averaging 2 replicates).

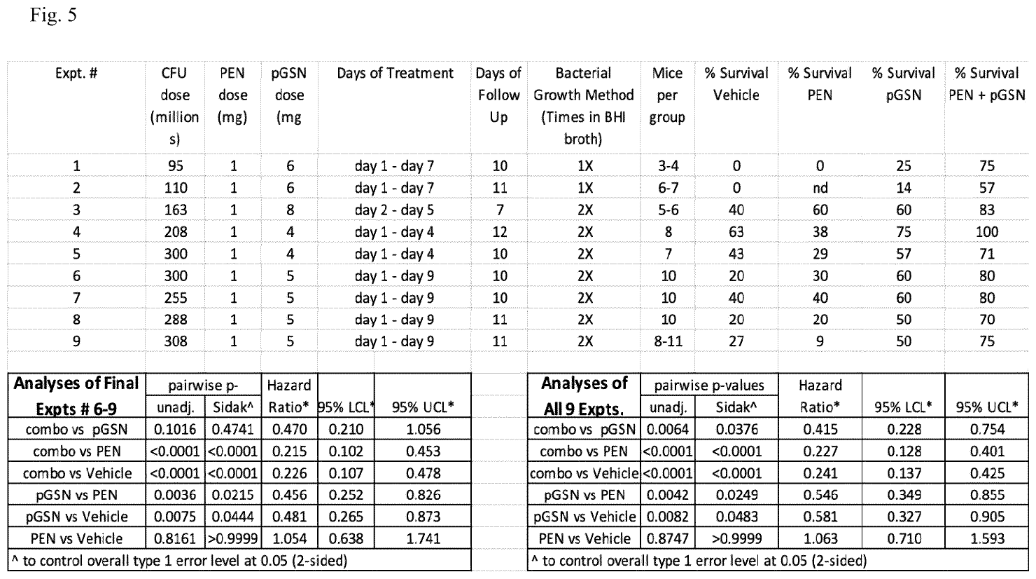

In vivo, treatment with a combination of penicillin and pGSN resulted in higher survival than with pGSN alone (FIG. 4A), but this was not was not statistically different when adjusted for multiple comparisons (p=0.47, see FIG. 5). Results of all survival experiments are presented in FIG. 5 and show that for each of the nine experiments, survival was highest in the pGSN+PEN group, followed by pGSN alone compared to either PEN or vehicle alone (and the pGSN+PEN combination was significantly better than pGSN alone). The FIG. 5 table provides results from nine experiments in which testing delayed administration of four treatments was assessed. Data from the final four experiments, which used essentially identical treatments and were representative of the overall results obtained in all nine studies, are shown in FIG. 4A-C. FIG. 5 provides details of all nine experiments, including pilot and range-finding trials. Column H shows a change in bacterial growth method obtained using a method for 2X growth in BHI broth for penicillin-resistant pneumococci (Restrepo AV et al., BMC Microbiol 2005; 5:34) for superior growth results. The survival differences were statistically significant, as determined by analysis of all the nine studies pooled using log rank analysis along with Sidak correction for multiple comparisons. Details of the results of statistical analysis of the final four experiments (#6-9) are summarized in FIG. 4A-C.

### Discussion

Studies were designed and configured to mimic the clinical situation where a subject presents after an infection is apparent. Therefore, the experiments were performed using a clinically relevant scenario of delaying administration until the mice had become visibly ill rather than the pre-or concurrent treatment used in prior studies (Yang Z. et al., Am J Physiol Lung Cell Mol Physiol 2015; 309: L11-6.). This design was used to evaluate the potential of pGSN to improve treatment outcomes. The key findings were that delayed pGSN treatment improved survival, either when used alone without an antibiotic or in combination with a suboptimal antibiotic to which the bacterial strain is highly resistant. The observed lowered bronchoalveolar neutrophil counts in infected pGSN-treated animals may reflect accelerated bacterial clearance by pGSN-stimulated resident macrophages, pGSN's inflammation-modulating activity, or both. With serotype 14, the ability to study longer delays before therapy was limited in pilot trials by the relatively high number of deaths by day 2 or 3 without treatment. Studies are performed to examine other antibiotic-resistant organisms in other model systems. Previous findings that pGSN enhances microbicidal function of macrophages against other bacteria (e.g., E. coli, F. tularensis LVS [Yang Z. et al., Am J Physiol Lung Cell Mol Physiol 2015; 309:L11-6.]) are encouraging in this regard, but direct testing is needed.

The totality of the data suggests a synergistic interaction of pGSN with penicillin treatment that had no effects by itself. However, this conclusion relies on pooled analysis of all the range-finding as well as final trials performed. When only the final four replicate studies (FIGS. 4A-C) are analyzed, the comparison is in the same direction but does not achieve statistical significance. Potentiation of penicillin effects on bacterial growth in vitro by concomitant rhu-pGSN was not observed. While not intending to be bound by any particular theory, these data suggest that antibacterial defenses enhanced by pGSN may be even more effective against bacteria that are slightly perturbed (but not killed) by penicillin. The mechanism merits future attention, especially if similar results are observed in other infections with resistant bacteria. In summary, rhu-pGSN can improve outcomes in a highly lethal pneumococcal pneumonia model when given after a clinically relevant delay, even in the setting of antimicrobial resistance. These findings support further evaluation of pGSN as an adjunctive therapy for serious antibiotic-resistant infections.

### Example 3

Studies were performed to evaluate effects of rhu-pGSN treatment to meropenem in highly lethal, multidrug-resistant P. aeruginosa pneumonia in a neutropenic mouse model.

### Methods

Production of rhu-pGSN

Recombinant human plasma gelsolin (rhu-pGSN), was produced in E. coli and subsequently lyophilized for reconstitution. Vehicle controls containing formulation components were used for the comparator mice.

### Bacteria Strain and Growth Conditions

P. aeruginosa UNC-D is a sputum isolate from a patient with cystic fibrosis. [Lawrenz MB, et al. Pathog. Dis. 73 (2015)]. Bacteria were cultured on trypticase soy agar (TSA) plates and in Lennox broth at 37° C. with shaking of broth cultures. Minimum inhibitory concentrations of the UNC-D strain are: ceftazidime [32 μg/ml], meropenem [8 μg/ml], imipenem [16 μg/ml], tobramycin [32 μg/ml], piperacillin [16 μg/ml], aztreonam [4 μg/ml], colistin [1 μg/ml], and fosfomycin [256 μg/mL]. Bacteria were prepared for animal challenge studies by culturing bacteria in Lennox broth overnight and washing the bacteria into 1X PBS before diluting to a final concentration based on OD 600-based estimates and a final 50 μl delivery dose. Bacterial inocula were confirmed by serial dilution and colony enumeration on TSA plates.

### Animal Respiratory Infection Model

The BALB/c infection model of P. aeruginosa UNC-D strain [Lawrenz M B, et al. (2015) Pathog. Dis. 73(5):ftv025] was specifically designed to test for adjunctive therapies that might result in improved efficacy of failing meropenem monotherapy against a multi-drug resistant (MDR) P. aeruginosa UNC-D strain resistant to several clinically important antibiotics including meropenem. Previous experience demonstrated this model is most informative when examining novel compounds using meropenem doses that provide approximately 50% mortality with meropenem treatment alone [Lawrenz MB, et al. (2015) Pathog. Dis. 73(5):ftv025]. Mice were housed and treated in accordance with standard animal experimentation guidelines at the University of Louisville. Briefly, female BALB/c mice were rendered neutropenic using cyclophosphamide injections (150 mg/kg) on days-5 and-3 prior to infection, typically resulting in ˜90% drop in the neutrophil counts. Approximately 105.5 CFU of UNC-D was directly instilled into the lungs by intubation- mediated intratracheal instillation. Meropenem (Hospira; Lake Forest, IL) was administered by subcutaneous injection beginning at 3 hours post-infection and q8h for 5 days.

To determine if rhu-pGSN adjunctive therapy improves the efficacy of meropenem, 12 mg/day of rhu-pGSN was administered by intraperitoneal injection of 0.3 ml at −24, −3, 3, 27, 51, 75, 99, and 123 hours post-infection. Mice were monitored for development of illness every 8 hours after infection for 7 days, including temperatures measured via transponders implanted subcutaneously prior to the initiation of the studies (BioMedic Data Systems; Seaford, DE). Moribund mice were humanely euthanized and scored as succumbing to the infection at the next time point. Tissues samples were harvested for bacterial counts and pathology as previously described [Lawrenz MB, et al. Pathog. Dis. 73 (2015)]. Mice surviving to 7 days were scored as surviving infection and euthanized; tissues were similarly processed. Lung histopathology was scored in a blinded fashion by a board-certified veterinary pathologist. A four-point, four-criteria system (inflammation; infiltrate; necrosis; and other, including hemorrhage) with a maximum score of 16 points was used to evaluate lung pathology. Points for each criterion were assigned based as no (0), minimal (1), mild (2), moderate (3), and severe (4) pathologic findings.

### Statistical Analyses

In total, 3 comparable experiments were independently performed using this model. Titration experiments were done when a new batch of meropenem was to be used to estimate the effective dose (ED)₅₀ for each lot of antibiotic prior to the formal experiments. Overall survival and survival with minimal lung injury (defined post hoc as histopathology scores ≤2) were tallied for the experiments overall and for experimental conditions where the meropenem-only control groups protected ≤50% of the mice. The 95% confidence intervals and p-values for differences in the proportions of surviving mice between treatment arms with and without rhu-pGSN were computed via normal approximation to the binomial distribution. For the individual experimental conditions where the mortality rate in the control meropenem group approximated 50% or more, survival curves were analyzed by the log rank test, temperature data were analyzed by two-way ANOVA, and bacterial burden and pathology scores were analyzed by one-way ANOVA with Tukey posttest multiplicity adjustment. The prespecified primary endpoint was survival 7 days post-infectious challenge. During analysis of these data, a “survival-plus” endpoint to examine survival with healthy lungs (histopathology score ≤2) was used as a clinically meaningful extension of a good outcome. Bacterial burden and temperature response were not included in this two-pronged composite because they were not direct measures of clinical improvement.

### Results

rhu-pGSN Improved Survival of Mice Infected with P. aeruginosa

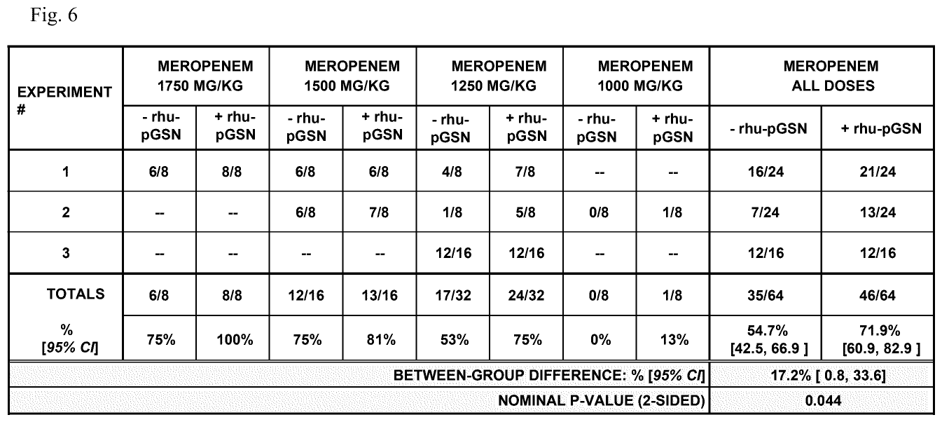

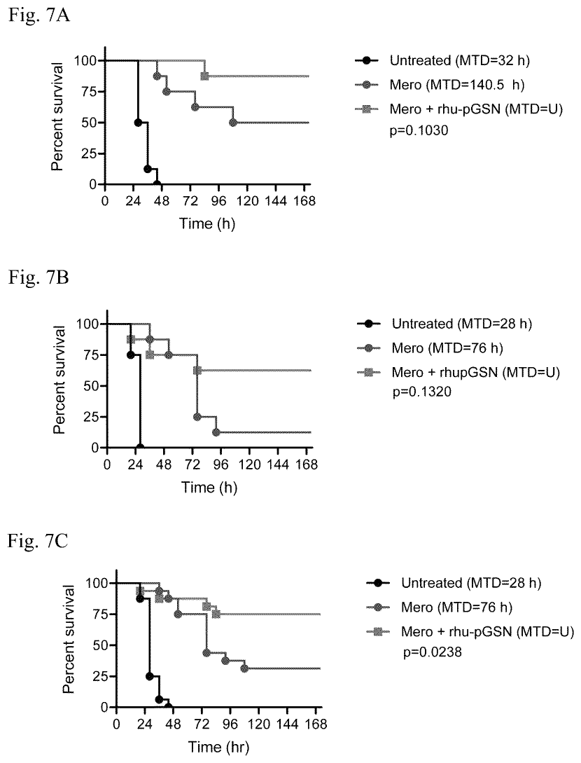

To determine whether rhu-pGSN could improve the efficacy of meropenem against pulmonary infection, female BALB/c mice were made neutropenic with cyclophosphamide (n=8), infected with MDR P. aeruginosa, and treated with varying doses of meropenem to determine the dose at which meropenem therapy begins to fail in this model (i.e., approached the EDso for meropenem). Mice were treated with the selected doses of meropenem with or without rhu-pGSN for 5 days post-infection and monitored for the development of moribund disease for 7 days post-infection (FIG. 6). In both experiments 1 and 2, treatment with 1250 mg/kg/day of meropenem resulted in ≤50% survival, indicating failure of meropenem treatment and allowing ascertainment of whether adjunctive therapy with rhu-pGSN could improve efficacy. Focusing on animals receiving this dose, addition of rhu-pGSN numerically increased the number of animals that survived to the end of each study (FIG. 7A-B). Combining the two sequential studies, 31% of the mice receiving meropenem alone survived for 7 days compared with 75% survival when mice were given meropenem with rhu-pGSN (Δ (95% confidence interval)=44% (13, 75); p=0.0238; FIG. 7C). A third experiment using a different lot of meropenem that demonstrated a higher than predicted meropenem efficacy (75% survival in meropenem only group) did not show a difference in survival rates between the treatment groups (FIG. 6).

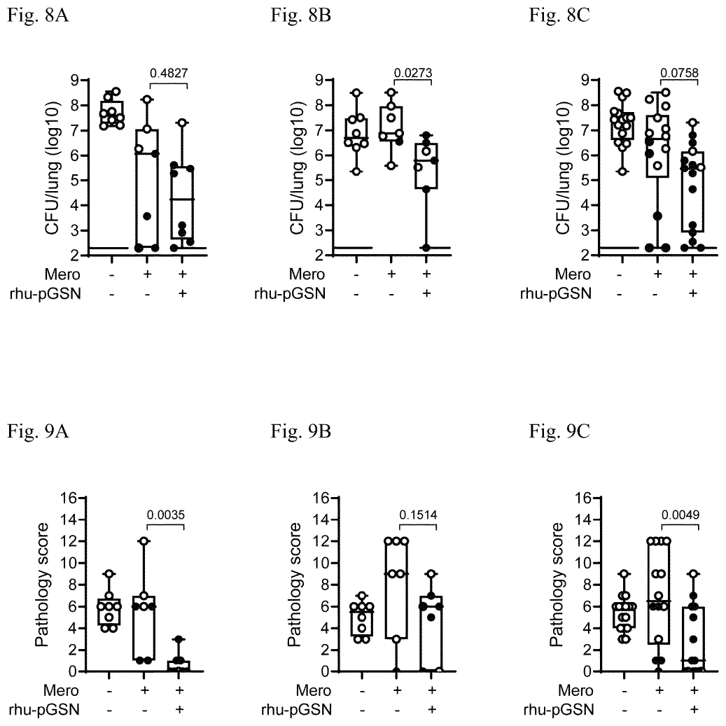

To ascertain if the increased survival with rhu-pGSN therapy was associated with decreased bacterial burden in the lungs, colony counts were determined from the lungs of mice receiving 1250 mg/kg/day at the time of euthanasia (FIG. 8A-C). A general trend was observed suggesting that rhu-pGSN improved control of bacterial burden in the lungs of infected mice compared to meropenem alone but a statistically significant difference in bacterial counts was only observed in the second study (p=0.0273).

Overall survival for all the dosing groups in the 3 experiments combined was 35/64 (55%) and 46/64 (72%) in mice treated with meropenem without or with rhu-pGSN, respectively [Δ (95% confidence interval)=17% (1, 34)]. Although treatment with adjunctive rhu-pGSN increased the efficacy of meropenem against pulmonary infection with P. aeruginosa, inhibition of bacterial proliferation in the lungs may only partially explain the observed benefit. Interestingly, it was observed that meropenem alone controlled spread from lung to spleen in both studies, but that pGSN allowed splenic colonization in some animals. While this observation was not significant in any study alone, combining data demonstrated a significant increase in splenic counts in pGSN-treated mice. In conjunction with improved survival, these observations were consistent with rhu-pGSN exerting an opsonic effect which enhanced splenic uptake.

rhu-pGSN Limits Acute Lung Injury

The lack of an unambiguous relationship between reduced bacterial loads in the lungs and increased survival in mice that received rhu-pGSN raised the possibility that rhu-pGSN protection might be mediated by alternative or additional mechanisms. Because pGSN modulates inflammation, the question of whether adjunctive rhu-pGSN therapy diminished lung injury was investigated in P. aeruginosa infected animals receiving 1250 mg/kg/day. Representative sections of lung tissue harvested from animals were blindly scored for pathology by a board-certified veterinary pathologist. Addition of rhu-pGSN to meropenem reduced host lung damage (FIG. 9A-B; p=0.0035 and p=0.1514, respectively). Combining the data from these two independent studies, the mean pathology score for mice receiving meropenem alone was 6.86, whereas the mean pathology score for mice that received both meropenem and rhu-pGSN was 2.53 (FIG. 9C; p=0.0049).

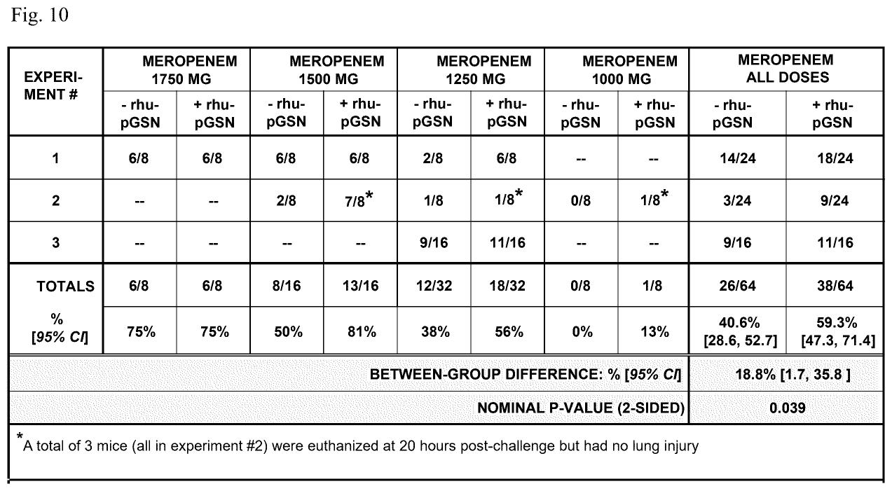

Based on these observations that rhu-pGSN protected against lung damage, the analysis was expanded to include mice receiving doses of meropenem above and below 1250 mg/kg/day. Overall survival of mice receiving different doses of meropenem for three individual experiments are shown in FIG. 6. Animals surviving infection for 7 days were grouped as either demonstrating near normal lung histology (pathology scores ≤2) or signs of lung pathology (pathology scores >2). Retrospectively using this criterion, overall survival with minor lung injury was found in 26/64 (41%) mice receiving only meropenem versus 38/64 (59%) mice given meropenem plus rhu-pGSN [Δ (95% confidence interval)=19% (2, 36)] (FIG. 10). To eliminate the noise generated by highly effective and ineffective meropenem doses, arbitrary but clinically reasonable exclusion limits of ≥75% and ≤25% were then imposed for the control survival rate. In this middle ground of responsiveness to meropenem alone, another exploratory post-hoc analysis yielded favorable outcomes (survival with near-normal lungs) in 12/32 (37.5%) with only meropenem and in 27/32 (84.4%) with the combination of meropenem and rhu-pGSN [Δ=47% (26, 68)].

Using surviving mice as the denominator, near-normal lung histopathology was found in 26/35 (74.3%) and 38/46 (82.6%), respectively, with meropenem treatment alone versus meropenem and rhu-pGSN combined therapy. These data together indicate that addition of rhu-pGSN may decrease lung injury caused by P. aeruginosa infection treated only with antibacterial agents.

### Plasma Gelsolin Speeds Resolution of the Host Systemic Response

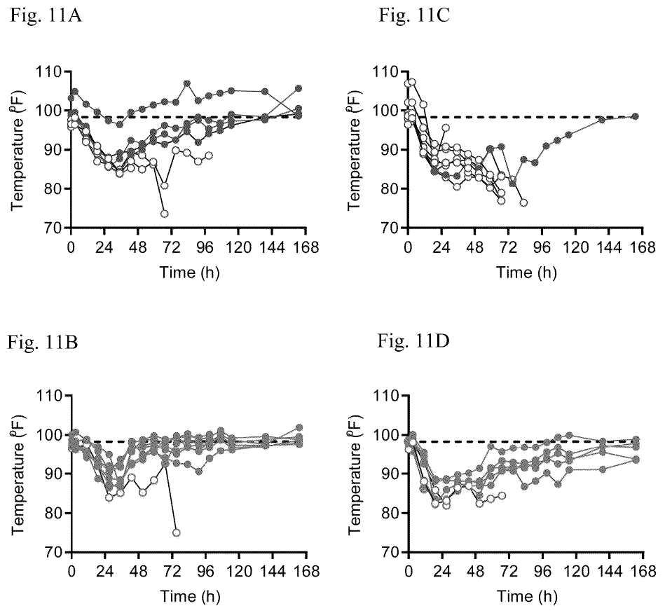

As part of monitoring disease progression, host temperature was followed over the course of infection. For this model, all mice tended to exhibit a steady decrease in body temperature within the first 24 hours of infection. For mice that received efficacious treatments, their temperatures eventually returned to normal, while the temperature of mice that received sub-efficacious treatments continued to decline [Lawrenz MB, et al. (2015) Pathog. Dis. 73(5):ftv025]. The time course of temperature normalization allowed assessment of differences in recovery rates between different treatments. Focusing on the dosing regimens approaching the targeted ED₅₀ for meropenem alone in these experiments, the question of whether pGSN sped the restoration of temperature homeostasis in mice surviving infection was investigated. In the two studies achieving a survival advantage, mice typically experienced an ˜10° F. decrease in body temperature within the first 24 hours after infection (FIG. 11A-D). Mice treated with meropenem alone who were to survive to Day 7 began to restore their body temperatures toward 95° F. within 3-5 days post-infection. In contrast, the restoration of host body temperature was much more rapid in mice treated with rhu-pGSN and meropenem, where survivor body temperatures returned to 95° F. by Day 2. Thus, adjunctive rhu-pGSN not only improved survival and lung pathology, but also accelerated systemic recovery of the host as measured by temperature curves. In the third experiment where a survival advantage with rhu-pGSN was not seen, no difference in the temperature course was observed between treatment arms.

### Discussion

rhu-pGSN improved survival when added to meropenem in an established murine model of severe multidrug-resistant P. aeruginosa pneumonia. Normalization of temperature in surviving mice generally occurred more rapidly with adjunctive rhu-pGSN therapy than with meropenem alone. Lungs from rhu-pGSN recipients generally had fewer viable bacteria. Furthermore, rhu-pGSN reduced the degree of acute lung injury in surviving animals, which potentially represents a clinically important advance in the treatment of serious bacterial pneumonia. Taken together, these findings suggest that survival advantage afforded by the addition of rhu-pGSN to meropenem treatment was likely due in large part a rhu-pGSN-mediated reduction in the bacterial load and severity of lung injury during the course of infection.

The first line of host defense against infection involves a focused inflammatory response. However, excessive local and systemic inflammation can be injurious to vital organs near and far from the primary infection site. As the acute injury recedes, pGSN promotes resolution of the inflammatory process and limits the resultant damage.

The possible benefits of adding rhu-pGSN treatment to meropenem were explored in highly lethal, multidrug-resistant P. aeruginosa pneumonia in a neutropenic mouse model. All mice died within ˜24 hours of infection without immediate antimicrobial therapy. Rhu-pGSN as sole treatment slightly prolonged average survival by ˜12 hours. To decide on the dose of meropenem that would yield >50% mortality, titration experiments were performed with each batch of antibiotic. Nonetheless, outcomes were not always predictable, leading to mortality rates ≤25% or ≥75% for the meropenem controls in some trials. Under such extreme conditions, possible benefits of adjunctive rhu-pGSN on outcome might be masked because the mice were either too sick or not sick enough. Nonetheless, rhu-pGSN given with meropenem was more efficacious than meropenem alone under most conditions.

These preclinical data further strengthen the growing body of evidence that rhu-pGSN as an adjunct to standard-of-care modalities might be effective in enhancing survival while limiting lung injury. Even with supraphysiological levels throughout the dosing interval, neither serious nor drug-related adverse events were observed in rhu-pGSN recipients given three consecutive days of therapy.

Using an established model of murine Gram-negative pneumonia, bacterial colony counts from alveolar lavage and histopathological lung injury scores at the time of euthanasia were higher in mice receiving meropenem alone compared to mice treated with meropenem and rhu-pGSN although there was considerable variability observed within and between experiments. Both mortality and parenchymal injury were lessened by the addition of rhu-pGSN to meropenem, most prominently in situations where meropenem alone was relatively ineffective.

### Example 4

### Methods

### Mouse Model of Influenza

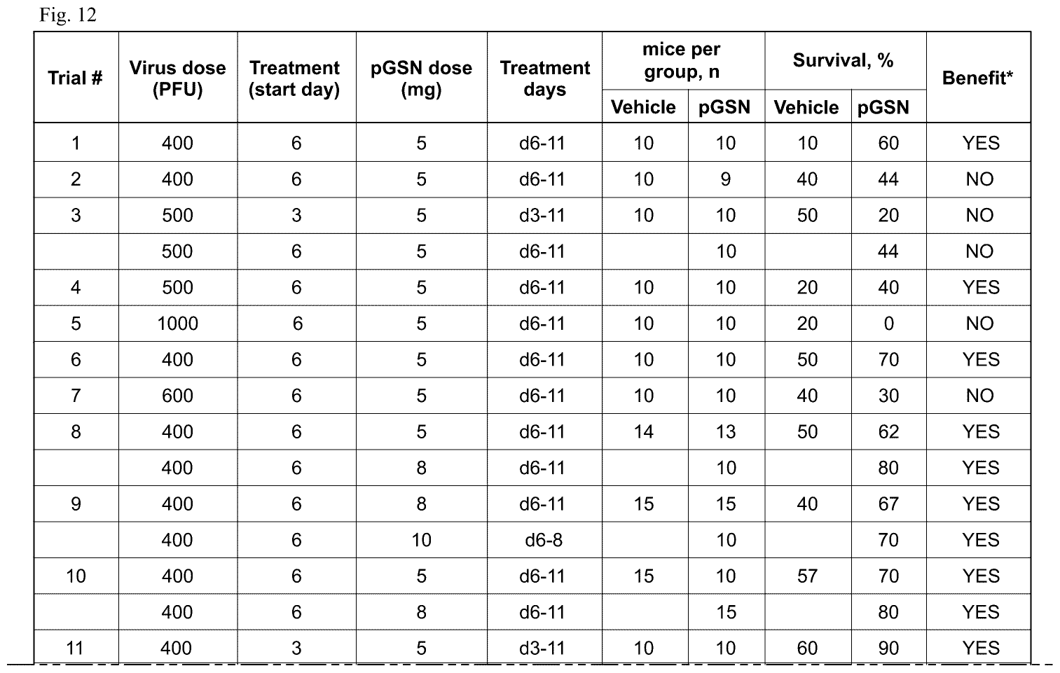

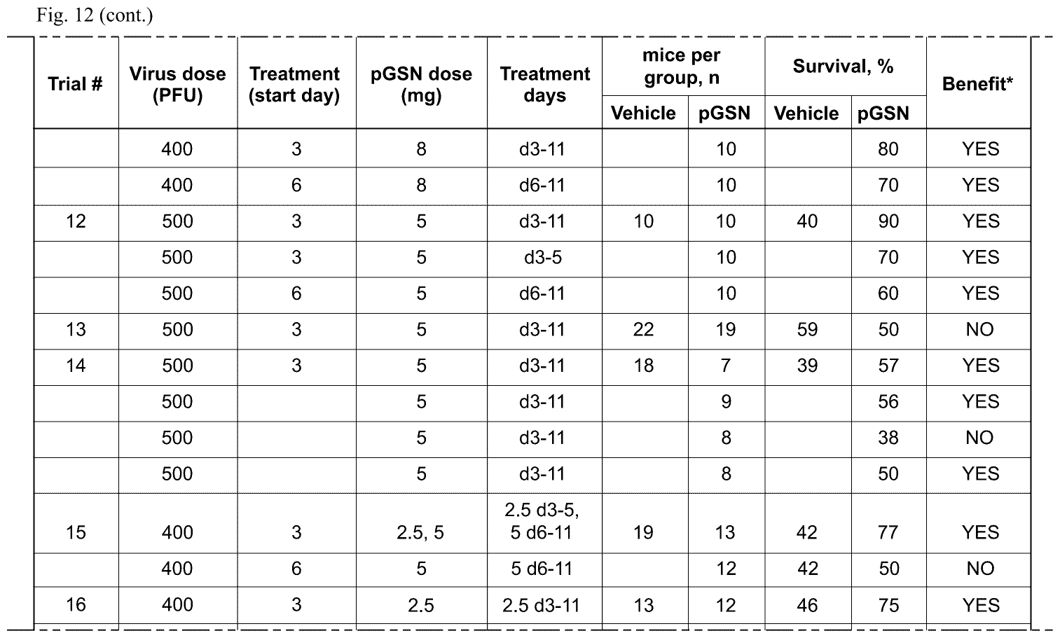

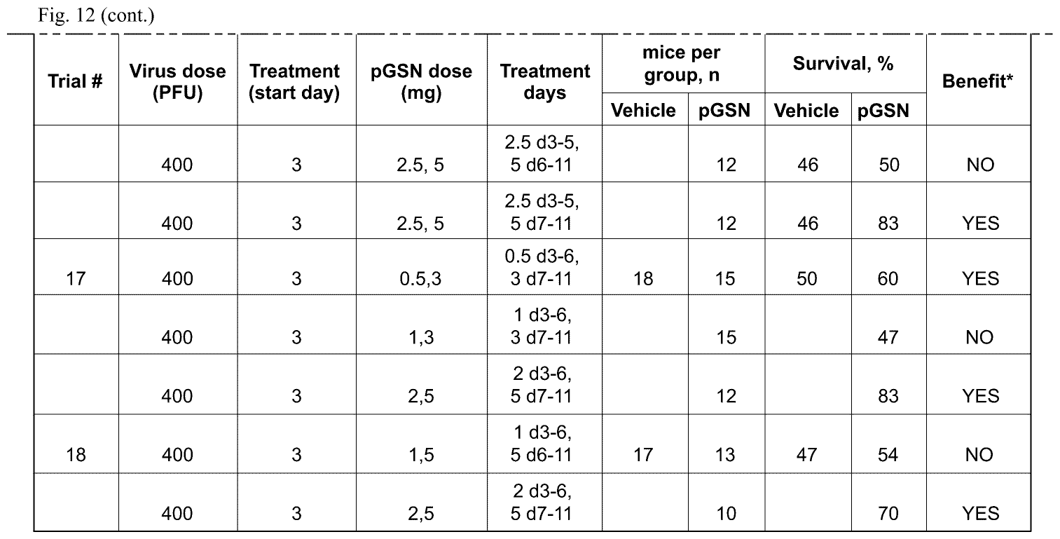

Normal 6- to 8-week-old male CD1 mice were obtained from Charles River Laboratories (Wilmington, MA). Only male mice were used due to budgetary and time limits. All mice arrived and were co-housed 1 week prior to the start of the experiments. Each trial used a separate batch of mice. A murine-adapted strain of H1N1 influenza virus, A/Puerto Rico/8/1934 (PR8), quantified as plaque-forming units (PFU) was procured from ViraSource (Durham, NC). Mice were anesthetized with 72 mg/kg ketamine plus 9.6 mg/kg xylazine administered via intraperitoneal injection. Mice then received an intranasal instillation of 2 5μl suspension of PBS containing virus (ranging from 400-1000 PFU depending on the trial) or vehicle alone. All infections were done at approximately the same time of day (starting at ˜10 AM). Initial titration identified 400 PFU as a dose that led to ˜60% mortality in vehicle-treated mice, and this dose was used in a majority of the trials (see FIG. 12). Most trials used at least 10 mice per group for the vehicle and pGSN treatment groups; details of the influenza dose, total number of mice, and their weights are provided in the tables in Underlying Data [Kobzik L: “Expanded Tables 1 & 2”. Harvard Dataverse, V1 2019. www.doi.org/10.7910/DVN/53GJY1].

### Treatments and Outcomes

Recombinant human pGSN (rhu-pGSN) was synthesized in E. coli and purified by Fujifilm Diosynth (Billingham, UK). Human rather than murine gelsolin was used based on prior demonstrations of function of rhu-pGSN in rodent models and because data with the human gelsolin will facilitate clinical translation efforts. Rhu-pGSN was administered daily to mice by subcutaneous injection starting on day 3 or 6 after infection, at doses ranging from 0.5-5 mg as detailed in Results. The mice were monitored for 12 days, measuring survival, changes in weight and overall morbidity using a composite index (i.e., 1 point each for hunched appearance, ruffled fur or partly closed eyes; 1.5 points for prolapsed penis or splayed hind quarter; 2 points for listlessness, with a maximum score of 8; the assessment was performed without blinding to treatment group) adapted from guidelines described previously [Burkholder T, et al., Current Protocols Mouse Biol. 2012; 2:145-65.] Weights and morbidity scores for the last day alive were carried forward for animals that did not survive.

### Lung Transcriptome Profiling

Lung tissue was obtained on days 7 and 9 after infection from mice treated with either vehicle or rhu-pGSN (dosed 2 mg per day starting on day 3 after infection, then increased to 5 mg per day on day 7). RNA was isolated using the RNAEasy mini-kit (Qiagen, Germantown, MD) according to manufacturer's instructions. RNA samples were analyzed using the Mouse DriverMap targeted gene expression profiling panel from Cellecta (Mountain View, CA). The Cellecta platform uses highly multiplexed RT-PCR amplification and next-generation sequencing (NGS) quantitation to measure expression of 4753 protein-coding and functionally significant mouse genes. The procedure detailed in the Cellecta User Manual, item 5.3 was followed to create amplified index libraries which were sequenced on an Illumina NextSeq 500 instrument. The sequencing data was converted to FASTQ format and then further analyzed using DriverMap Sample Extraction software. This produced a raw data matrix file of counts for each sample in columns aligned to the 4753 gene panel.

### Statistical Analysis

Data were analyzed using Prism (GraphPad Software) or SAS (SAS Institute) software. Differences in Kaplan-Meier survival curves were analyzed using a log-rank test with Sidak adjustment for multiple comparisons. A Breslow-Day test for homogeneity of the pGSN versus vehicle comparison across studies yielded p>0.2, indicating homogeneity could not be rejected and supporting the overall comparison across studies, which was carried out via the log-rank (Mantel-Cox) test stratified by trial. For other measurements, differences between groups were examined by ANOVA. The transcriptome profiling results scaled to normalize column counts, were converted to log2 counts (after addition of 0.1 to all cells to eliminate zero values) and then analyzed using Qlucore software (Lund, Sweden). Further analysis of gene set enrichment was performed using tools (Panther version 14.118 and MetaCore (version 19.3, Clarivate Analytics, Philadelphia, PA)) that allow evaluation using a custom background gene list (i.e., the ˜4700 genes measured using the Cellecta DriverMap platform).

### Results

Effect of rhu-pGSN on Survival

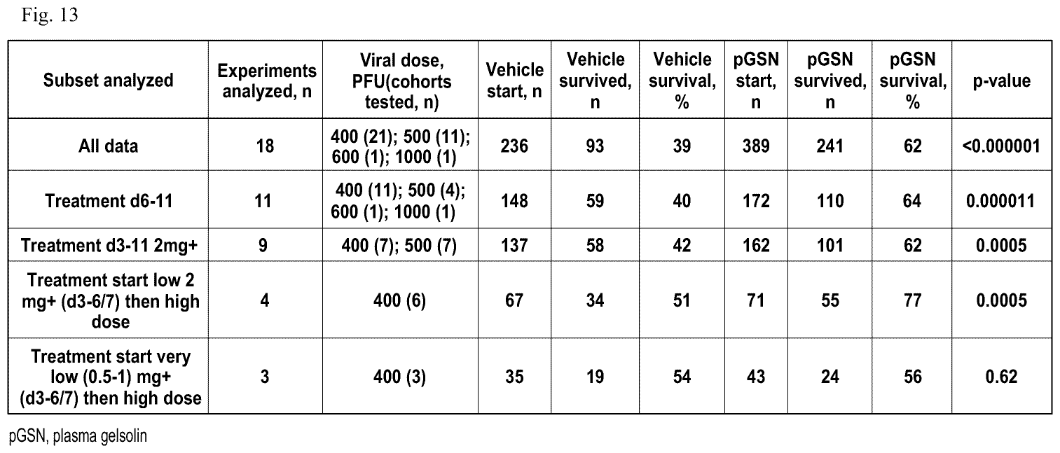

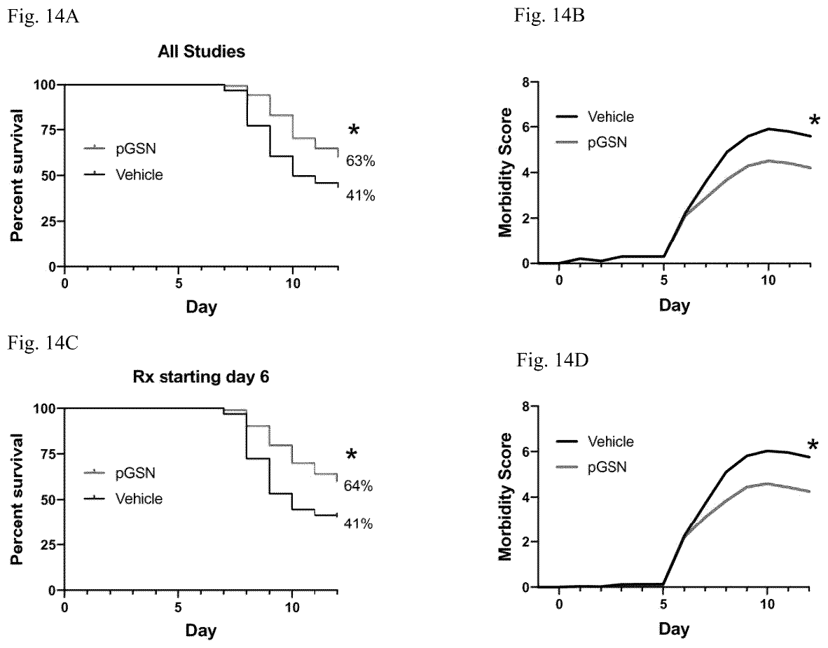

A variety of dose and timing regimens were tested to evaluate the potential of rhu-pGSN to improve outcomes, conducting a total of 18 trials that are tabulated in FIG. 12 and summarized in FIG. 13. To mimic likely clinical usage, mice were not treated until several days post-challenge.

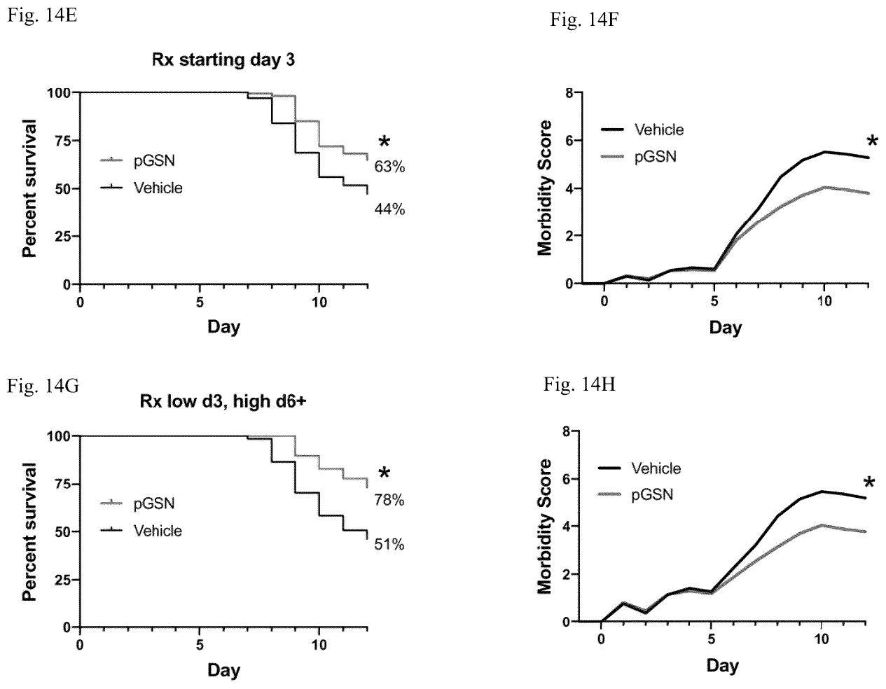

A main finding was that delayed treatment with rhu-pGSN resulted in significant improvement in the survival of mice (FIG. 14A-H). All studies combined yielded 39% (93/236) surviving mice treated with vehicle and 62% (241/389) surviving mice treated with pGSN on day 12 (p=0.000001, FIG. 14A). Improved survival was observed whether the delayed treatment was started on day 6 (FIG. 14C) or day 3 after infection (FIG. 14E, 14G). Similarly, compared to vehicle treatment, rhu-pGSN resulted in decreased morbidity scores (FIG. 14B, 14D, 14F, 14H). In contrast, no statistically significant difference in weight loss or recovery (in surviving animals) was consistently observed in the experiments summarized in FIG. 14A-H. The sole exception was found in the trials testing a dose regimen of initially low (>2 mg rhu-pGSN on days 3-6/7, then 5 mg through day 11). The latter set of trials led to weights (compared to day 0) at the end of study of 81.4±4.7% in vehicle-treated mice versus 85±2.6% in pGSN-treated mice (p<0.0001, summary of 4 trials, see also FIG. 12 and FIG. 13, and more detailed tabulation of all experiments in Extended data [Kobzik L: “Expanded Tables 1 & 2”. Harvard Dataverse, V1 2019. www.doi.org/10.7910/DVN/53GJY1]. A beneficial effect of rhu-pGSN was observed in a majority but not all of the 18 individual trials (FIG. 12, see Discussion).

### Transcriptome Profiling

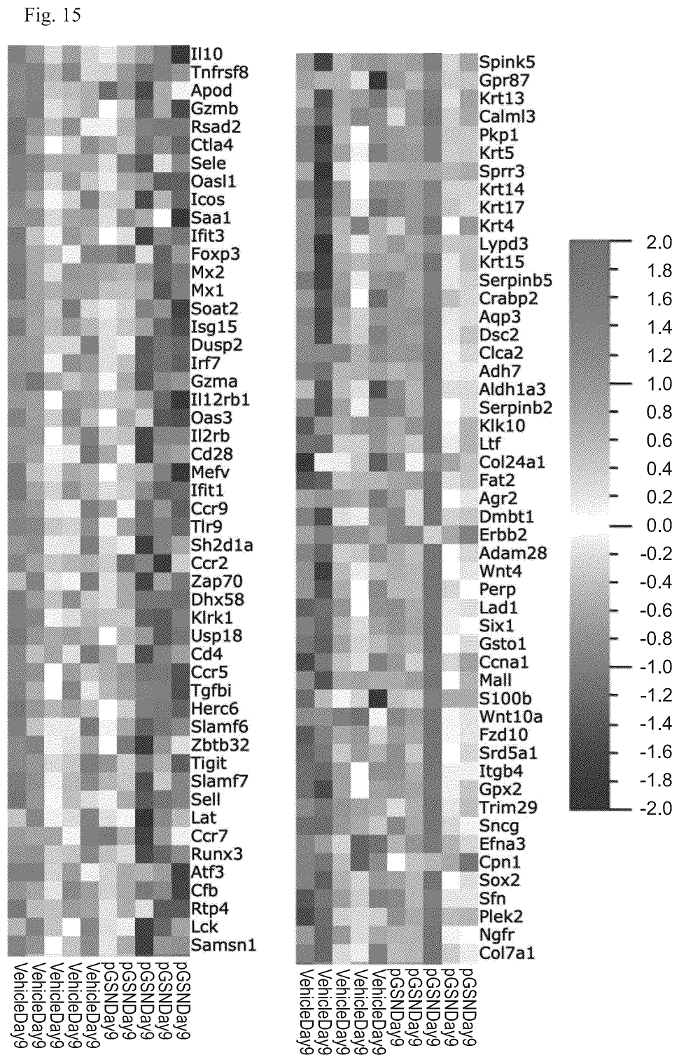

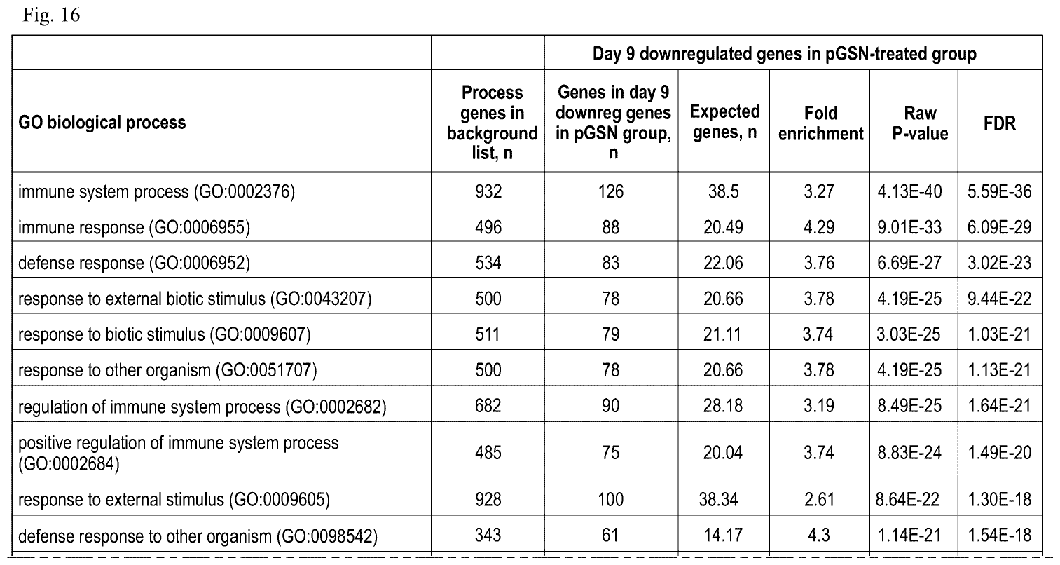

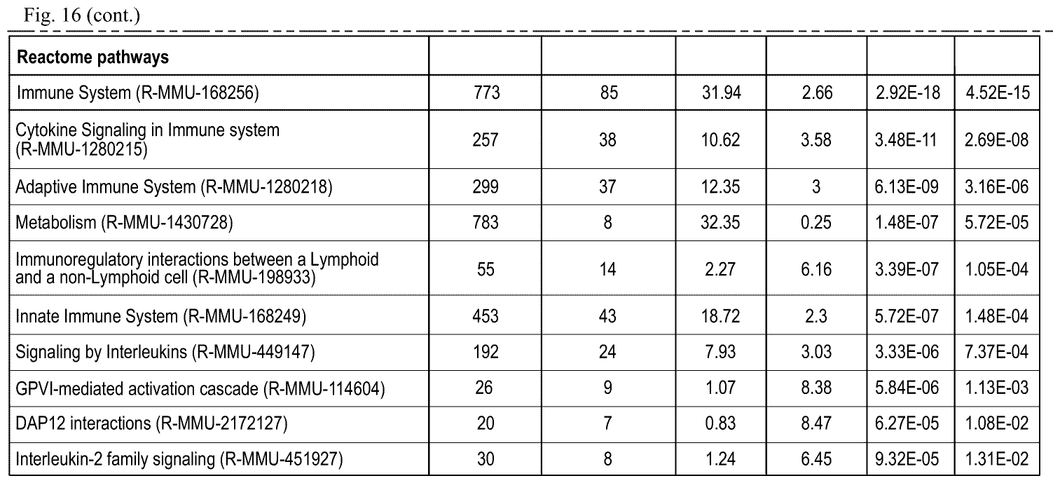

To evaluate whether rhu-pGSN treatment modified the transcriptome profile [see Harvard Dataverse: Expanded Tables 1 & 2. //doi.org/10.7910/DVN/53GJY116] of infected lungs, lung tissue was harvested just before (day 7) and after (day 9) the usual onset of mortality (day 8) in this model (n=5 per group per day). Per protocol, the rhu-pGSN dose was increased in this experiment on day 7, between the 2 time points selected for profiling. Comparison of lung samples obtained at day 7 from vehicle-treated and rhu-pGSN-treated mice showed no significant differences. In contrast, analysis of day 9 samples identified 344 differentially expressed genes in the rhu-pGSN-treated group, comprised of 195 down-regulated and 149 up-regulated genes. The top 50 up-and down-regulated genes are shown in FIG. 15, which is notable for the many cytokine and immune-related genes prominent among those down-regulated in the rhu-pGSN-treated group (including IL10, IL12rb, CTLA4, and CCRs9, 7 and 5, among others). Gene enrichment analysis of the full down-regulated gene list was performed using the Panther online analysis tool to query GO Ontology or Reactome databases. The main findings were a reduction of expression of biological processes linked to immune and inflammatory responses, or release of cytokine and other cellular activators. The top 10 most significant processes/pathways are shown in FIG. 16. Analysis using a different gene enrichment analysis software tool (MetaCore) produced similar results. Analysis of the up-regulated gene list identified enrichment of processes related to tissue morphogenesis and epithelial/epidermal cell differentiation (consistent with repair of influenza-mediated damage, see Discussion). Details of the DriverMap gene list, the differentially expressed genes identified, and the full results of gene enrichment analyses using the down-and up-regulated gene lists to query the Panther and MetaCore databases are presented in worksheets 2-15 in a spreadsheet available in Extended data [Kobzik L: Harvard Dataverse, V1 2019. www.doi.org/10.7910/DVN/8HBFD7]. Data on experimental groups in studies described herein, are shown in Table 1 and Table 2 of The Harvard Dataverse: Expanded Tables 1 & 2. //doi.org/10.7910/DVN/53GJY116, which also describes additional variables, such as weight, and statistical analyses. Additional data from experiments described herein are provided at: NCBI Gene Expression Omnibus: Transcriptome profiling of lung tissue from influenza-infected mice treated with plasma gelsolin. Accession number GSE138986; //identifiers.org/geo:GSE138986.

### Discussion

Studies were performed to evaluate the potential of rhu-pGSN to improve outcomes in severe influenza using a clinically relevant scenario of delaying initiation of treatment. A key finding was that delayed pGSN treatment significantly improved survival, either when used starting on day 3 or even starting as late as day 6 after infection. In addition to the impractically of initiating earlier therapy right after infection (as opposed to the onset of severe symptoms) in patients, the delay was implemented so as not to interfere with the immediate immune response to influenza given the detrimental consequences observed in some experimental models.

Some limitations merit discussion. The first is the experimental variability observed. Treatment with rhu-pGSN increased survival in a majority of the experiments conducted, but not in all of them. For some of the negative trials, were believed the result of factors such as, but not limited to: technical issues with the virus stock, variation in instillation method, insufficient initial rhu-pGSN dose in the ‘low dose then high dose’ trials, etc. To the extent possible the methods were adjusted to reduce these potential sources of variability.

Experimental variables were also manipulated, to examine for example, whether treatment as late as day 6 vs day 3 after onset of infection be effective and to assess other variables in the studies. Ultimately, beneficial effects were observed whether the survival analysis included all the trials (FIG. 14A, B) or those using treatment starting at day 6 or day 3 (FIG. 14C-H).

Mice were only followed for 12 days when euthanasia was performed on surviving mice. Because the survival curves were still potentially declining, the ultimate mortality rate could not be confidently ascertained. However, the time to death at a minimum was prolonged with rhu-pGSN over placebo treatment.

Notably, rhu-pGSN did not rescue all of the mice dying from influenza in the experimental model, though the results indicated a significant survival benefit. Given the goal of identifying a novel therapy for severe influenza, it was interpreted that the results obtained in mice without the supportive fluid, additional therapeutic agents (for example but not limited to antiviral agents), and respiratory care given to hospitalized patients, supports a conclusion that methods would confer similar benefits as well as synergistic benefits in clinical settings. The results suggest that combination therapy of administering a gelsolin agent at a suitable time following infection, with standard therapeutics such as antiviral medications, offers a greater survival advantage.

In summary, rhu-pGSN can improve outcomes in a highly lethal murine influenza model when given after a clinically relevant delay. These findings are consistent with the benefits seen in models of pneumococcal pneumonia. The modes of action for pGSN involve host responses and do not seem to depend on the specific type of pathogen. The experimental results support use of gelsolin as an adjunctive therapy for severe influenza and other viral infections in humans and other mammals.

### Example 5

Additional studies are performed in which synergistic amounts of a gelsolin agent and an antiviral agent. In certain studies oseltamivir phosphate, zanamivir, peramivir or baloxavir marboxil is the antiviral administered to the subject. The gelsolin agent is administered in a delayed-dose method as described above herein. Effective amounts of the antiviral agent and the gelsolin agents are administered to a subject having or suspected of having a viral infection, such as one of Influenza A, B, C, or D and the effective amounts result in a synergistic therapeutic effect against the viral infection in the subject. The synergistic therapeutic effect improves one or more characteristics of the viral infection in the subject by a greater amount than an improvement in the one or more characteristics in a control, wherein the control does not receive a treatment that includes administration of synergistically effective amounts of the gelsolin agent and the antiviral agent.

### Equivalents

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1-156. (canceled)

157. A method of treating a subject having or suspected of having a bacterial infection resistant to an antibacterial agent, the method comprising:
administering to the subject having or suspected of having the bacterial infection resistant to the antibacterial agent, effective amounts of each of a gelsolin agent and the antibacterial agent, wherein administration of each of the gelsolin agent and the antibacterial agent has a greater therapeutic effect against the bacterial infection than when only the gelsolin agent is administered.

158. The method of claim 157, wherein the effective amount of the antibacterial agent is below a maximum tolerated dose (MTD) of the antibacterial agent, the MTD being a highest possible but still tolerable dose level of the antibacterial agent for the subject.

159. The method of claim 157, wherein:
the therapeutic effect against the bacterial infection is greater than a control therapeutic effect against the bacterial infection; and
the control therapeutic effect is a sum of a therapeutic effect of the antibacterial agent on the bacterial infection plus a therapeutic effect of the gelsolin agent on the bacterial infection when each of the antibacterial agent and the gelsolin agent is administered without the other.

160. The method of claim 157, wherein the gelsolin agent comprises a gelsolin molecule, a functional fragment thereof, or a functional derivative thereof.

161. The method of claim 160, wherein the gelsolin molecule is a plasma gelsolin (pGSN) or a recombinant gelsolin molecule.

162. The method of claim 157, wherein administration of each of the gelsolin agent and the antibacterial agent decreases a minimum effective dose (MED) of the antibacterial agent in the subject.

163. The method of claim 157, wherein administration of each of the gelsolin agent and the antibacterial agent reduces a level of the bacterial infection in the subject compared to a level of infection in the absence of administering each of the gelsolin agent and the antibacterial agent.

164. The method of claim 157, wherein administration of each of the gelsolin agent and the antibacterial agent increases the subject's likelihood of survival compared to a likelihood of survival in the absence of administration of each of the gelsolin agent and the antibacterial agent.

165. The method of claim 157, wherein administration of each of the gelsolin agent and the antibacterial agent reduces a level of lung pathology in the subject compared to a level of lung pathology in the absence of administration of each of the gelsolin agent and the antibacterial agent.

166. The method of claim 157, wherein the bacterial infection is caused by a type of Pseudomonas aeruginosa.

167. The method of claim 166, wherein the antibacterial agent comprises a carbapenem class antibiotic.

168. The method of claim 167, wherein the carbapenem class antibiotic is meropenem.

169. The method of claim 157, wherein the bacterial infection is caused by a type of Streptococcus pneumoniae.

170. The method of claim 169, wherein the antibacterial agent comprises a β-lactam antibiotic.

171. The method of claim 170, wherein the β-lactam antibiotic is penicillin.

172. The method of claim 157, wherein the bacterial infection is caused by one or more of a gram-positive bacterium, a gram-negative bacterium, a tuberculosis bacillus, a nontuberculous mycobacterium, a spirochete, an actinomycete, an Ureaplasma species bacterium, a Mycoplasma species bacterium, and a Chlamydia species bacterium.

173. The method of claim 157, wherein administration of the gelsolin agent and the antibacterial agent are independently selected from oral, sublingual, buccal, intranasal, intravenous, intramuscular, intrathecal, intraperitoneal, subcutaneous, intradermal, topical, rectal, vaginal, intrasynovial, and intra-ocular administration.

174. The method of claim 157, wherein the subject is a mammal.

175. The method of claim 157, wherein the gelsolin agent is not administered the day the subject is infected with the bacterial infection.

176. The method of claim 175, wherein the gelsolin agent is administered at least 3 days after infection of the subject with the bacterial infection.
